(19) United States
(12) Patent Application Publication
(10) Pub. No.: US 2011/0172455 A1
(43) Pub. Date: Jul. 14, 2011

(54) PROCESS FOR PREPARING CINACALCET AND PHARMACEUTICALLY ACCEPTABLE SALTS THEREOF

(75) Inventors: Venkateswaran Srinivasan Chidambaram, Chennai (IN); Ramkaran Prajapaty, Haryana (IN); Perminder Singh Johar, Punjab (IN); Ekta Sharma, Haryana (IN); Lalit Wadhwa, Haryana (IN)

(73) Assignee: IND-SWIFT LABORATORIES LIMITED, Chandigarh (IN)

(21) Appl. No.: 13/003,140

(22) PCT Filed: Jul. 6, 2009
(86) PCT No.: PCT/IN2009/000401

§ 371 Date: Jan. 7, 2011

(30) Foreign Application Priority Data
Jul. 8, 2008 (IN) 1635/DEL/2008
Jan. 15, 2009 (IN) 69/DEL/2009
Jan. 15, 2009 (IN) 70/DEL/2009

(51) Int. Cl. C07C 271/10; C07C 211/15

(52) U.S. Cl. 560/28; 564/373

(57) ABSTRACT

The resent invention rovides a novel rocess for re arin cinacalcet of formula I and pharmaceutically acceptable salts thereof and process of purification. The present invention also provides novel nitrogen protected synthetic intermediates useful in the process of the present invention. Further, the present invention provides a novel substituted carbamate impurity and process of preparation thereof.

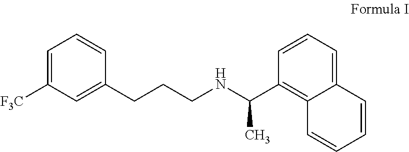

### FIELD OF THE INVENTION

The present invention provides a novel process for preparing cinacalcet of formula I,

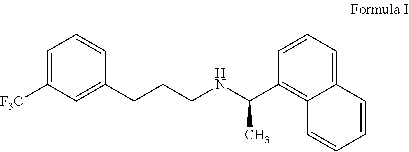

and its pharmaceutically acceptable salts thereof.

The present invention also provides novel nitrogen protected synthetic intermediates useful in the process of the present invention. Particularly, the present invention provides an industrially advantageous process for the preparation of cinacalcet hydrochloride.

The present invention provides an impurity of cinacalcet, substituted carbamate of cinacalcet. The present invention further provides a process for the purification of cinacalcet and its salts thereof.

### BACKGROUND OF THE INVENTION

Cinacalcet of formula I, and cinacalcet hydrochloride are novel calcimimetic agents that modulate the extra cellular calcium sensing receptor by making it more sensitive to the calcium-suppressive effects on parathyroid hormone and is chemically known as N-[1-(R)-(−)-(1-naphthyl)ethyl]-3-[3-(trifluoromethyl)phenyl]-1-aminopropane.

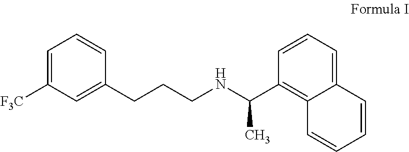

It is used in the treatment of primary and secondary hyperparathyroidism. Hyperparathyroidism is characterized by high levels of circulating calcium due to an increased secretion of parathyroid hormone by one or more of the parathyroid glands. Hyperparathyroidism can lead to osteoporosis; patients with renal failure suffering from secondary hyperparathyroidism have for example an increased risk of renal bone disease, soft-tissue calcifications and vascular disease. Calcium receptor-active molecules like cinacalcet and its pharmaceutically acceptable salts thereof were disclosed in PCT publication WO 1994/18959, U.S. Pat. Nos. 6,211,244, 6,313,146, 6,031,003, 6,001,068, 6,011,884, 5,962,314, 5,858,684, 5,841,368, 5,763,569 and 5,688,938 etc. U.S. Pat. No. 6,211,244 discloses the process for the preparation of calcium receptor-active molecules like einacalcet, but does not provide any example for the preparation of cinacalcet and its pharmaceutically acceptable salt thereof.

The method disclosed in the above patents for the preparation of these compounds includes the reductive amination of a commercially available aldehyde or ketone with a primary amine in the presence of sodium cyanoborohydride or sodium triacetoxyborohydride.

Alternatively, some compounds were prepared from the condensation of a primary amine with an aldehyde or ketone in the presence of titanium (IV) isopropoxide. The resulting imine intermediate were then reduced in situ by the action of sodium cyanoborohydride, sodium borohydride, or sodium triacetoxyborohydride and the intermediate enamines were then catalytically reduced using palladium dihydroxide on carbon.

Various compounds were prepared by a diisobutylaluminium hydride (DIBAL-H) mediated condensation of an amine with a nitrile. The resulting imine intermediate was reduced in situ by the action of sodium cyanoborohydride or sodium borohydride. The intermediate alkene was reduced by catalytic hydrogenation in ethanol using palladium on carbon. Further, the compounds obtained by the above processes were converted to corresponding hydrochloride salts by treatment of the free base with hydrogen chloride gas in ether or hexane in combination with hydrogen chloride gas. The processes disclosed here involve the use of expensive reagents. In addition, the compounds prepared there are purified by the column chromatography.

Drugs of future 2002, 27(9), 831-836 discloses a process for the preparation of cinacalcet according to general process disclosed in U.S. Pat. No. 6,211,244. The process involves the reaction of 1-acetylnaphthalene with 3-[3-(trifluoromethyl)phenyl]propylamine in presence of titanium isopropoxide to produce an imine which on treatment with methanolic sodium borohydride gives racemic base which is then resolved by chiral chromatography.

U.S. Pat. No. 7,250,533 discloses a process for the synthesis of cinacalcet by first converting hydroxyl moiety of 3-(3-trifluoromethylphenyl)propanol into a compound containing good leaving group and further combining the same with (R)-1-naphthylethylamine in the presence of a base in an organic solvent to obtain cinacalcet according to the following scheme:

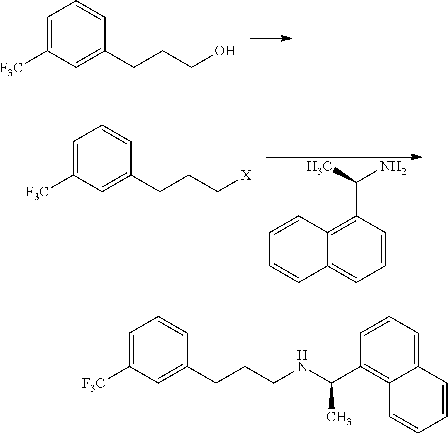

Intermediate, 3-(3-trifluoromethylphenyl)propanol is prepared by the heck coupling of 1-bromo-3-trifluoromethylbenzene with ethylacrylate to give unsaturated ester, followed by reduction to give corresponding saturated alcohol.

PCT publication WO 2007/127445 discloses a process for the preparation of cinacalcet by the condensation of reactive derivative of 3-(3-trifluoromethylphenyl)propanoic acid with (R)-1-naphthyl ethylamine to give N-[(1R)-1-(1-naphthyl)ethyl]-3-[3-(trifluoromethyl)phenyl]propanamide, which is then reduced to give cinacalcet and its pharmaceutically acceptable salts as shown in the following scheme:

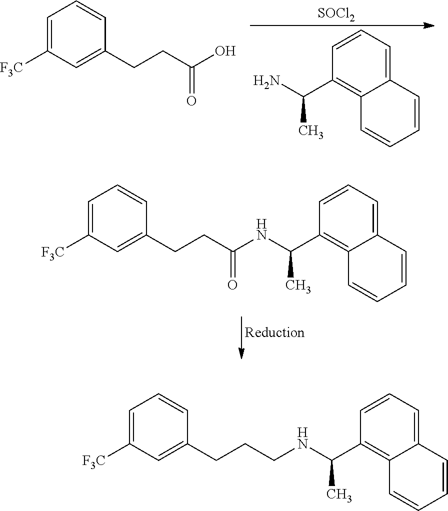

Similar process is also disclosed in PCT publications WO 2008/035381, WO 2008/058235; Indian application no. 555/MUM/2007; articles, Tetrahedron Letters 2008 49(1) 13-15 and Synthetic Communications 2008 38 (10) 1512-1517.

PCT publication WO 2007/127449 discloses a process for the preparation of cinacalcet by condensation of 3-bromotrifluorotoluene with an allylamine of following formula

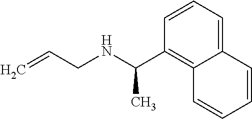

in the presence of a catalyst and at least one base to obtain unsaturated cinacalcet which is then reduced to give cinacalcet.

PCT publication WO 2008/063645 discloses a process for the preparation of cinacalcet by condensing a compound of following formula,

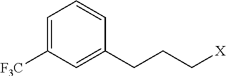

wherein X is C₁-C₃ alkyl sulfonate, substituted and non-substituted C₆-C₁₀ aryl sulfonate or halogen with (R)-1-naphthylethylamine using minimal amount of solvent and optionally, in the presence of a base.

PCT publication WO 2008/068625 discloses a process for the preparation of cinacalcet by reductive amination of 3-(3-trifluoromethylphenyl)propanal with (R)-1-naphthylethylamine in the presence of sodium triacetoxyborohydride.

As discussed above, most of the prior art processes involve the use of reagents such as titanium isopropoxide and diisobutylaluminium hydride, which are expensive and have to be handled in large volume when the process is employed on large scale. These moisture sensitive and pyrophoric reagents require special handling. One of the processes involve the use of ethylacrylate which is a known carcinogen, highly flammable, may cause violent reaction on exposure to moisture and unstable to oxidizing agent. Use of compound like ethylacrylate is not advisable due its instability to above conditions. Use of column chromatography for the purification; chiral chromatography for the isolation of chiral compounds, use of expensive and harmful reagent make the processes known in the prior art not amenable to an industrial scale up.

It is well known that any synthetic compound, such as cinacalcet and pharmaceutically acceptable salts thereof can contain extraneous compounds or impurities. These impurities may be, for example, starting materials, by-products of the reaction, products of side reactions, or degradation products. Impurities in any active pharmaceutical ingredient like cinacalcet, are undesirable and in extreme cases, might even be harmful to a patient being treated with a dosage form containing the API and therefore must be absent in the final API like cinacalcet hydrochloride. Therefore, an active pharmaceutical compound must be free from such process impurities, side products or degradation impurities before it is formulated.

In addition to purification of the active ingredient, to remove impurities, analysis of the impurities present is also necessary. So that such impurities present in the final API can be analyzed at certain stages during processing of an API, such as cinacalcet and pharmaceutically acceptable salts thereof. U.S. Pat. No. 7,294,735 discloses a carbamate impurity of cinacalcet and process of its preparation by the reaction of methanesulfonic acid 3-(3-trifluoromethyl-phenyl)-propyl ester with (R)-naphthyl ethylamine in the presence of base in a solvent at elevated temperature. The patent also discloses a process for the purification of cinacalcet hydrochloride to remove cinacalcet carbamate impurity. The process involves the purification of cinacalcet containing 3 to 6 percent carbamate impurity in a solvent selected from acetone, linear or branched-chain C₂₋₈ ether, and mixture thereof or with water which is then converted to cinacalcet hydrochloride. The patent discloses another method for the removal of cinacalcet carbamate impurity by column chromatography and high pressure liquid chromatography which is not amenable on industrial scale.

U.S. Pat. No. 7,294,533 discloses a process for the purification of cinacalcet to remove starting material, (R)-1-naphthylethylamine as impurity. The process disclosed in the patent first involves the salt formation of cinacalcet by the acidification of cinacalcet in a solvent and then neutralization to give cinacalcet which further have to be converted to cinacalcet hydrochloride, as active compound used for the formulation is cinacalcet hydrochloride. This seems to be a lengthy process for obtaining cinacalcet hydrochloride free from (R)-1-naphthylamine impurity. The patent also exemplifies the purification of crude cinacalcet with column chromatography which is very cumbersome techniques and not applicable for the commercial production.

PCT publication WO2008/58236 discloses the purification of cinacalcet hydrochloride by dissolving cinacalcet hydrochloride in nitrile solvent followed by the addition of anti solvent to the above solution.

Therefore, there is an urgent need in the art to develop a process for the preparation of cinacalcet and its pharmaceutically acceptable salts thereof, which is industrially applicable, does not involve the use of harmful and expensive reagent like ethylacrylate and diisobutylaluininium hydride. Chemical purity is also very much important in the field of pharmaceuticals therefore there is also a need to obtain cinacalcet hydrochloride in high purity. In order to achieve high chemical purity, cinacalcet hydrochloride must be free from known and unknown impurities. So there is need to develop a purification process that provides cinacalcet hydrochloride free from impurities or impurities acceptable amounts. In addition, identification of the impurities, that may be present in the samples of cinacalcet and cinacalcet pharmaceutically acceptable salts, is also required so that their process for their removal can be chosen after identification of their structure as well as nature. Thus, the present invention fulfills the need of the art and provides an industrially advantageous process for the preparation of cinacalcet and its pharmaceutically acceptable salts using novel intermediates. The process of the present invention is cost effective, eco-friendly, commercially viable as well as reproducible on industrial scale and meets the needs of regulatory agencies. The present invention provides process for the purification of cinacalcet and salts thereof, as well as provide a novel impurity, substituted carbamate impurity, so that its presence can be easily checked in a sample of cinacalcet or pharmaceutically acceptable salts thereof.

### OBJECT OF THE INVENTION

It is the principal object of the present invention to provide an efficient and novel process for the preparation of cinacalcet and its pharmaceutically acceptable salts thereof, which is unique with respect to its simplicity, cost effectiveness and convenience to operate on industrial scale. Another object of the present invention is to provide novel process for the preparation of synthetic intermediates.

Another object of the present invention is to provide novel synthetic intermediates that play a crucial role in the preparation of cinacalcet and its pharmaceutically acceptable salts thereof.

Another object of the invention is to provide processes for the purification of cinacalcet and its salts. Another object of the invention is to provide a process for the purification of cinacalcet to remove certain identified and unidentified impurities.

Another object of the present invention is to provide industrially advantageous processes for the purification of cinacalcet hydrochloride that yields cinacalcet hydrochloride having impurities in acceptable limits or preferably free from impurities.

Still another object of the invention is to provide a substituted carbamate impurity of cinacalcet and process of substituted carbamate impurity.

Yet another object of the invention is to provide a process for the removal of the substituted carbamate impurity from the sample of cinacalcet hydrochloride.

Yet another object of the invention is to provide cinacalcet hydrochloride having substituted carbamate impurity 0.03 to 0.15% by HPLC.

### SUMMARY OF THE INVENTION

According to one aspect, the present invention provides a novel and industrially advantageous process for the preparation of cinacalcet of formula I,

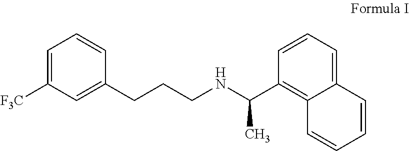

and its pharmaceutically acceptable salts thereof,
which comprises the steps of:
(a) providing a compound of formula II;

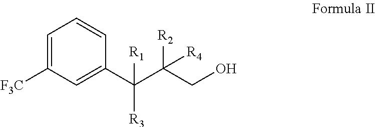

wherein R₁, R₂, R₃ and R₄ are hydrogen; or R₁, R₂ together, form a double bond provided R₃ and R₄ are hydrogen or R₃, R₄ together, form a double bond provided R₁ and R₂ are hydrogen; or R₁, R₂, R₃ and R₄ all are combined together to form triple bond
(b) converting the hydroxyl group of compound of formula II into a good leaving group to obtain compound of formula III;

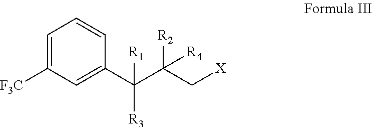

wherein R₁, R₂, R₃ and R₄ are as defined above and X is a good leaving group, preferably selected from halide such as chloro, bromo, iodo; or sulphonyloxy functional group of general formula —SO₂R′ wherein R′ is selected from straight chain or branched C₁₋₁₀ alkyl group; substituted or unsubstituted C₁₋₁₀ aryl group; substituted or unsubstituted C₁₋₁₀ heteroaryl group having one or more hetero atoms selected from nitrogen, sulfur or oxygen
(c) condensing the compound of formula III with the compound of formula IV,

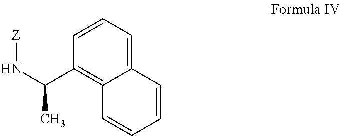

wherein Z is an amine protecting group
in presence of a suitable base to prepare a compound of formula V; and

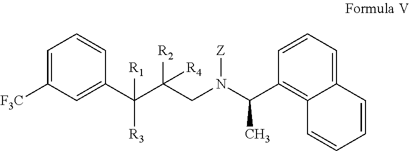

wherein R₁, R₂, R₃, R₄ and Z are as defined above
(d) converting the compound of formula V to cinacalcet of formula I and its pharmaceutically acceptable salts thereof.

According to another aspect, the present invention provides a novel process for the preparation of compound of formula II by reducing the compound of formula VI,

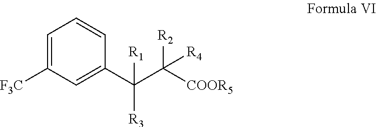

wherein R₁, R₂, R₃ and R₄ are as defined above and R₅ can be selected from hydrogen, alkyl or any suitable activating group
using a suitable reducing agent.

According to another aspect, the present invention provides novel intermediate of formula V including salts, hydrates, solvates, racemates, enantiomers, polymorphs, derivatives thereof and process for preparing the same and their conversion to cinacalcet and pharmaceutically acceptable salts thereof.

According to yet another aspect, the present invention provides a substituted carbamate impurity of cinacalcet having formula VII.

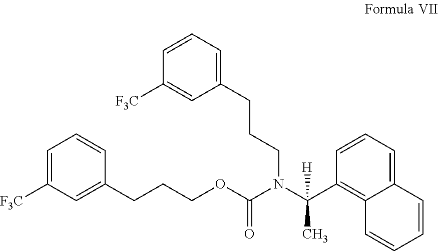

According to another embodiment, the present invention provides cinacalcet hydrochloride having substituted carbamate impurity in amount of 0.03% to 0.15% as measured by HPLC and process of its preparation.

The process comprises the step of:
(a) reacting a compound of formula IV,

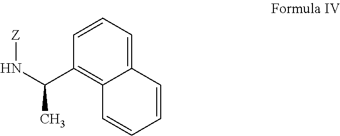

wherein Z is selected from hydrogen or a functional group of general formula —COOR″ wherein R″ is selected from straight chain or branched C₁₋₃ alkyl group; substituted or unsubstituted aryl group
with a compound of formula IIIa

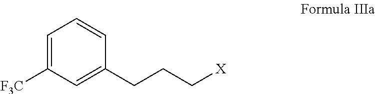

wherein X is as defined above
in the presence of a base in a solvent to form a compound of formula Va

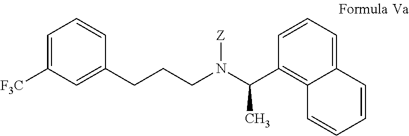

wherein Z is as defined above
having substituted carbamate impurity of formula VII in a range of 2 to 20% by HPLC;
(b) treating the compound of formula Va with a source of hydrogen chloride in a solvent to form cinacalcet hydrochloride;
(c) purifying the cinacalcet hydrochloride with a suitable solvent; and
(d) isolating cinacalcet hydrochloride having substituted carbamate impurity of formula VII less than 0.15% by HPLC.

According to one another aspect, the present invention provides a process for the purification of cinacalcet, comprising the steps of:
(a) providing a solution of cinacalcet in a solvent;
(b) adding silica gel to the solution;
(c) removing the solvent from the mixture by distillation or evaporation;
(d) adding a solvent to the residue;
(e) filtrating the solvent from the mixture;
(f) optionally, repeating steps (d) to (e);
(g) recovering pure cinacalcet from the filtrate; and
(h) optionally, converting cinacalcet to cinacalcet hydrochloride.

According to another aspect, the present invention provides a process for purification of cinacalcet hydrochloride, which comprises:
(a) slurrying cinacalcet hydrochloride in a suitable solvent; and
(b) isolating cinacalcet hydrochloride from the reaction mixture.

According to another aspect, the present invention provides a process for purification of cinacalcet hydrochloride, comprising the steps of
(a) providing a solution of cinacalcet hydrochloride in a solvent;
(b) washing the solution with water or aqueous solution of a suitable acid;
(c) distilling the solvent;
(d) slurrying the resulting residue in a suitable solvent; and
(e) isolating cinacalcet hydrochloride from the reaction mixture.

According to yet another aspect, the present invention provides a process for purification of cinacalcet hydrochloride, comprising
(a) providing a solution of cinacalcet hydrochloride in a suitable solvent;
(b) neutralizing the solution with a suitable base;
(c) removing the solvent from the solution;
(d) optionally, isolating cinacalcet;
(e) adding solvent to resulting residue;
(f) treating the solution with lithium aluminium hydride;
(g) quenching the reaction mixture;
(h) removing the solvent to obtain a residue; and
(i) treating the resulting residue in a solvent with a source of hydrogen chloride to form cinacalcet hydrochloride.

### DETAILED DESCRIPTION OF THE INVENTION

As described herein “all the intermediates, impurity as well as final product” includes salts, hydrates, solvates, racemates, enantiomers, polymorphs, derivatives thereof.

The present invention provides a novel and industrially advantageous process for the preparation of cinacalcet of formula I and its pharmaceutically acceptable salts thereof starting from compound of formula II including isomers or mixture thereof.

According to one aspect of the invention, hydroxy group of compound of formula II, including isomers or mixture thereof is converted to a good leaving group in presence of an activating reagent in a suitable solvent to form a compound of formula III including isomers or mixture thereof.

Generally, the compound of formula II in a suitable solvent is reacted with an activating reagent containing a good leaving group in a suitable solvent and maintaining the reaction mixture at a temperature of 0° C. to 180° C. The reaction temperature may vary depending upon the nature of activating agent. The time for obtaining the compound of formula III depend upon the quantity as well as nature of starting compound, activating reagent and reaction conditions, preferably reaction is maintained for half an hour to 24 hours; more preferably till the completion of the reaction. Activating reagent containing the good leaving group is generally a conjugate base. Activating reagent includes, but not limited to thionyl halide, aliphatic or aromatic sulfonyl halide, phosphorous halides, phosphorous oxyhalide and the like, preferably the activating reagent is thionyl bromide or thionyl chloride, methanesulfonyl chloride, benzenesulfonyl chloride, 4-nitrobenzenesulfonyl chloride or p-toluenesulfonyl chloride, phosphorus trichloride, phosphorous pentachloride, phosphorous oxychloride, phosphorous tribromide and the like. Solvent includes, but not limited to water, halogenated solvents such as dichloromethane, chloroform; C₂₋₈ ether such as isopropyl ether, methyl tert-butyl ether; C₃₋₈ aromatic and aliphatic hydrocarbon such as toluene, xylene, ethyl benzene; C₂₋₅ nitrile such as acetonitrile; C₃₋₈ ketone such as acetone, ethyl methyl ketone; methyl isobutyl ketone; amide solvents such as dimethyl formamide, dimethylacetamide, N-methylpyrrolidone; and the like or mixture thereof. The reaction can be preferably carried out in anhydrous or hydrous conditions. Anhydrous conditions can be created by employing anhydrous starting material, reagents as well as solvent or moisture can be removed by azeotropic distillation of water. It is advantageous to add a suitable base to the reaction mixture. Base can be an organic or an inorganic base. Organic base includes tertiary amines selected from triethylamine, N,N-diisopropylethyl amine, pyridine, and the like or combination thereof. Inorganic base includes but not limited to alkali or alkaline metal hydroxide, carbonate, bicarbonate and the like or combinations thereof, preferably the base can be selected from sodium hydroxide, potassium hydroxide, sodium carbonate, potassium carbonate, sodium bicarbonate, lithium hydroxide and the like.

In a preferred aspect, the compound of formula II wherein the R₁, R₂, R₃ and R₄ are hydrogen, has the structure of formula IIa including isomers or mixture thereof,

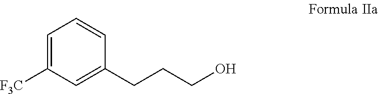

is converted to corresponding compound of formula IIIa including isomers or mixture thereof, by the similar process as described above.

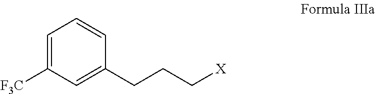

wherein X is a good leaving group

In another preferred aspect, compound of formula II wherein the R₁, R₂, together, form a double bond provided R₃ and R₄ are hydrogen or R₃, R₄, together, form a double bond provided R₁ and R₂ are hydrogen, has the structure of formula IIb including isomers or mixture thereof,

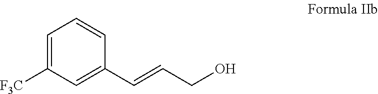

which is converted to corresponding compound of formula Mb, by the similar process as described above.

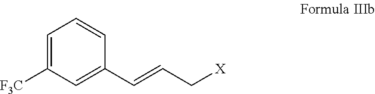

wherein X is as defined above.

In yet another preferred aspect, compound of formula II wherein the R₁, R₂, R₃ and R₄ all are combined together to form triple bond, has the structure of formula IIc including isomers or mixture thereof,

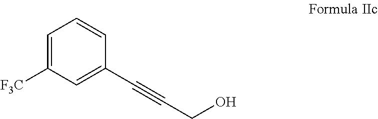

which is converted to corresponding compound of formula IIIc, by the similar process as described above.

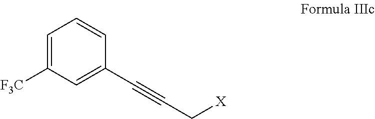

wherein X is as defined above.

According to another aspect, the present invention provides a process for the preparation of compound of formula IIIc.

Generally, the compound of formula IIIa can be prepared by the reduction of compound of formula IIIb or IIIc with a suitable reducing agent. Similarly, the compound of formula IIIb can be prepared by the selective reduction of compound of formula IIIc with a suitable reducing agent. The reduction reaction can be performed by catalytic hydrogenation (hydrogen over a metal catalyst). The metal catalyst includes, but not limited to transition metal, transition metal on support (where support can be carbon or barium sulfate), organometallic compounds of transition metal (homogenous catalyst), or other transition metal derivative or platinum dioxide and the like. The transition metal includes, but not limited to palladium, platinum, rhodium, ruthenium or nickel and the like. The hydrogen pressure employed in the reaction can be from 1 to 5 atmospheres. The hydrogenation is carried till the completion of the reaction, preferably for 1 to 24 hours. Reducing agents include, but not limited to borane complexes such as borane-tetrahydrofuran, borane-dimethylsulfide, borane amine, borane lewis base, borane-triphenylphosphine and the like; hydride transfer reagent. The reducing agents, MBR₆H or MAlR₆H that can be used with or without cocatalysts include, but not limited to cobalt or nickel derivatives and with or without ligands like dimethylglyoxime and the like (wherein M can be metal like alkali metal or alkaline earth metal or transition metal or a suitable metal and R₆ can be any ligand selected from alkoxy, RN, (R)₂N, (R)₃N, RCOO, RS, CN and the like; R is selected from substituted or unsubstituted alkyl, alkenyl, alkynyl, substituted or unsubstituted aryl, substituted or unsubstituted aralkyl and the like); or other appropriate reducing reagent as mentioned in comprehensive organic transformation by Richard C. Larock. The suitable solvent for the reduction reaction can be selected depending upon the reaction conditions and nature of reducing agent. Suitable solvents includes, but not limited to water; C₁₋₅ alcohol such as methanol, ethanol, isopropanol, tert-butanol, n-butanol; C₅₋₈ aliphatic or aromatic hydrocarbon such as toluene, xylene, ethyl benzene; C₃₋₈ ester such as ethyl acetate; C₂₋₈ ether such as isopropyl ether, tert-butyl ether; and the like or mixture thereof.

According to another aspect, the present invention provides a process for the preparation of compound of formula V,

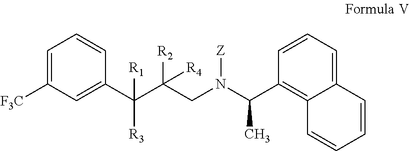

wherein R₁, R₂, R₃ and R₄ are as defined above; Z is an amine protecting group and can be selected from allyl; substituted allyl; linear, branched or cyclic C₁₋₈ alkyl; substituted linear, branched or cyclic C₁₋₈ alkyl; linear, branched or cyclic C₁₋₈ alkenyl; substituted linear, branched or cyclic C₁₋₈ alkenyl; linear, branched or cyclic C₁₋₈ alkynyl; substituted linear, branched or cyclic C₁₋₈ alkynyl; —CN; —SO₂R″; —COOR″ wherein R″ can be alkyl, alkenyl, alkynyl, or aryl; —CONR′″R′″ wherein R″′ and R″″ can be same or different and individually selected from alkyl, alkenyl, alkynyl, or aryl; or and the like; all the above groups can be substituted at carbon with a group selected from alkyl, alkoxy or aryl and like, preferably Z is selected amongst carbobenzyloxy, p-methoxybenzyl carbonyl, tert-butyloxycarbonyl, 9-fluorenylmethyloxycarbonyl, benzyl, p-methoxybenzyl, 3,4-dimethoxybenzyl, benzyloxycarbonyl group, p-methoxyphenyl, tert-butyldimethylsilyl; other sulfonyl such as p-nitrobenezenesulfonyl, methanesulfonyl, p-toluenesulfonyl, benzenesulfonyl group, and the like by the condensation of the compound of formula III with a compound of formula IV in presence of a base.

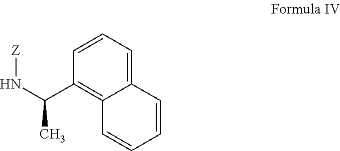

wherein Z is as defined above

Generally, the condensation reaction can be performed in the presence of a base in a suitable solvent at a temperature of about 0 to 100° C. for few minutes to several hours. The reaction temperature and time can vary depending upon the nature of the protecting group; preferably, reaction is carried out till the completion of the reaction. Solvent includes, but not limited to water, C₁₋₅ alcohol; C₃₋₈ ketone; C₅₋₈ aliphatic or aromatic hydrocarbon; C₃₋₇ ester; C₂₋₈ ether; C₂₋₅ nitrile; amide solvents such as dimethylformamide, N-methylpyrrolidone, dimethylacetamide; aprotic solvents such as dimethylsufoxide, and the like or mixture thereof. Suitable bases can be an organic or an inorganic base. Organic bases include, but not limited to tertiary amines; RM or RMgX (wherein R can be alkyl or aryl and M can be alkali or alkaline earth metal); or alkoxide of alkali or alkaline earth metal. Inorganic bases, includes but not limited to alkali or alkaline earth metal hydride, or hydroxide or carbonate or bicarbonate; or MNH₂ or MNSiR₇ (wherein M can be alkali metals and R₇ can be C₁₋₈ aliphatic or aromatic hydrocarbons and the like); or organometallic bases with or without additives. Optionally, a phase transfer catalyst can be added to the reaction mixture. Phase transfer catalyst includes, quaternary ammonium compounds: benzyl trimethylammonium chloride and bromide, cetyl trimethylammonium bromide, phosphonium compounds or synthetic resins, tetrabutylammonium bromide or chloride; benzyltriethylammonium chloride; tetrabutylammonium hydroxide; tricaprylmethylammonium chloride, dodecyl sulfate, sodium salt, such as sodium lauryl sulfate; tetrabutylammonium hydrogensulfate; hexadecyltributylphosphonium bromide; hexadecyltrimethyl ammonium bromide or resin amberlite IRA-410 and the like. Phase transfer catalyst may be present in an amount of about 0.05 to about 1.0 mol, preferably 0.05 to 0.5 mol equivalents. The compound of formula V can be isolated from the reaction using a suitable conventional method depending upon the nature of the compound of formula V.

In a preferred embodiment of the present invention, the compound of formula V wherein the R₁, R₂, R₃ and R₄ are hydrogen has structure formula Va,

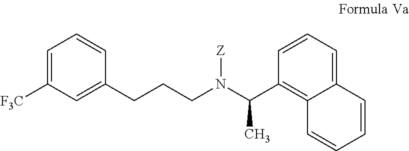

wherein Z is as defined above
may be prepared by the condensation of compound of formula IIIa with compound of formula IV and further forms the inventive part of the invention.

In a more preferred embodiment of the present invention, the compound of formula Va wherein Z is p-nitrobenzenesulfonyl has structure of formula Va-1,

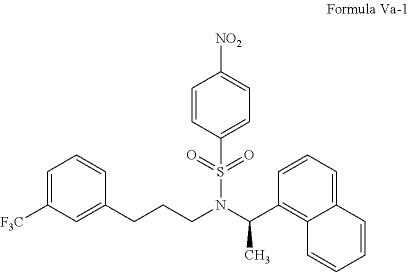

may be prepared by the condensation of compound of formula IIIa with compound of formula IV-1.

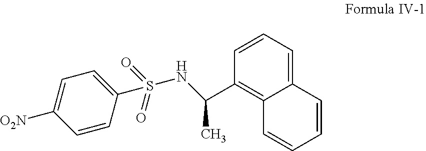

Generally, the process involves the condensation reaction of compound of formula IIIa (wherein x is as defined above) with compound of formula IV-1 in the presence of a base in a suitable solvent at a temperature of about 10 to 100° C. for few minutes to several hours, preferably till the completion of the reaction. Solvent includes, but not limited to water, C₁₋₅ alcohol such as methanol, ethanol, isopropanol; C₃₋₈ ketone such as acetone, methyl isobutyl ketone, methyl ethyl ketone; C₅₋₈ aliphatic or aromatic hydrocarbon such as toluene, xylene, ethyl benzene; C₃₋₇ ester such as ethyl acetate; C₂₋₈ ether such as isopropyl ether, methyl tert-butyl ether; C₂₋₅ nitrile such as acetonitrile; amide solvents such as dimethylformamide, N-methylpyrrolidone, dimethylacetamide and aprotic solvents such as dimethylsufoxide, and the like or mixture thereof. Suitable bases can be an organic or an inorganic base. Organic bases include, but not limited to tertiary amines; RM or RMgX (wherein R can be alkyl or aryl and M can be alkali or alkaline earth metal); or alkoxide of alkali or alkaline earth metal. Inorganic bases, includes but not limited to alkali or alkaline earth metal hydride, or hydroxide or carbonate or bicarbonate; or MNH₂ or MNSiR₇ (wherein M and R₇ are as defined above); or organometallic bases with or without additives, preferably base is selected from such as potassium carbonate, sodium hydroxide, sodium carbonate, sodium bicarbonate, potassium bicarbonate, potassium hydroxide; and the like. It is advantageous to perform the reaction optionally in the presence of phase transfer catalyst that includes, but not limited to quaternary ammonium compounds: benzyl trimethylammonium chloride and bromide, cetyl trimethylammonium bromide, phosphonium compounds or synthetic resins, tetrabutylammonium bromide or chloride; benzyltriethylammonium chloride; tetrabutylammonium hydroxide; tricaprylmethylammonium chloride, dodecyl sulfate, sodium salt, such as sodium lauryl sulfate; tetrabutylammonium hydrogensulfate; hexadecyltributylphosphonium bromide; hexadecyltrimethyl ammonium bromide or resin amberlite IRA-410 and the like, preferably benzyltriethylammonium chloride. The phase transfer catalyst may be present in an amount of about 0.05 to about 1.0 mol, preferably 0.05 to 0.5 mol equivalents. The compound of formula Va-1 can be isolated from the reaction mixture using suitable techniques known in the art such as removal of solvent from the reaction mixture by evaporation, distillation and the like.

Specifically, condensation of compound of formula IIIa (wherein x is p-toluenesulfonyl) with compound of formula IV-1 is accomplished in the presence of a base and optionally in the presence of phase transfer catalyst in a suitable solvent. The reaction can preferably be conducted at a temperature of room temperature to 90° C. and it takes about 10-15 hours for the completion of reaction. After completion of reaction, the reaction mass is cooled and neutralized using water and dilute acid solution. Thereafter, the solvent can be removed by distillation and another solvent may be added to resulting residue to isolate the solid compound. Another solvent can be selected from aliphatic or aromatic hydrocarbon such as n-heptane, cyclohexane, n-hexane; ether such as isopropyl ether and the like or mixture thereof. Preferably, another solvent may be selected from any solvent in which the desired product is having no solubility or less solubility.

In still another more preferred embodiment of the present invention, the compound of formula Va wherein Z is tert-butyloxycarbonyl has structure of formula Va-2

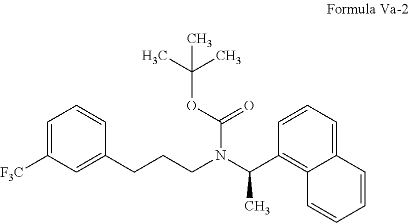

may be prepared by the condensation of compound of formula IIIa with compound of formula IV-2.

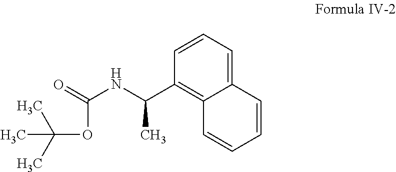

Typically, the process involves the condensation reaction of compound of formula IIIa (wherein X is as defined above) with compound of formula IV-2 in the presence of a base in a suitable solvent at a temperature of about 0 to 100° C. for few minutes to several hours, preferably till the completion of the reaction. Solvent includes, but not limited to water, C₁₋₅ alcohol such as methanol, ethanol, isopropanol, tert-butanol; C₃₋₈ ketone such as acetone, methyl isobutyl ketone, methyl ethyl ketone; C₅₋₈ aliphatic or aromatic hydrocarbon such as toluene, xylene, ethyl benzene; C₃₋₇ ester such as ethyl acetate, butyl acetate; C₂₋₈ ether such as tetrahydrofuran, isopropyl ether, methyl tert-butyl ether; C₂₋₅ nitrile such as acetonitrile; amide solvents such as dimethylformamide, N-methylpyrrolidone, dimethylacetamide and aprotic solvents such as dimethylsulfoxide and the like or mixture thereof. There is no limit on the nature of the solvent used for reaction, provided they have no effect on other functionalities. The base used for the reaction can be selected from an organic such as to tertiary amines; RM or RMgX (wherein R can be alkyl or aryl and M can be alkali or alkaline earth metal); or alkoxide of alkali or alkaline earth metal; or an inorganic base that alkali or alkaline earth metal hydride, or hydroxide or carbonate, or alkoxide or bicarbonate; or MNH₂ or MNSiR₇ (wherein M and R₇ are as defined above); or organometallic bases with or without additives, preferably base is selected from such as potassium hydroxide, potassium tertiary butoxide, sodium tertiary butoxide, sodium hydride, sodium hydroxide and the like. Optionally the reaction can be conducted in the presence of phase transfer catalyst which can be selected from the list as described above. The compound of formula Va-2 can be preceded as such for the next step or isolated from the reaction mixture. The isolation may be carried out using a suitable techniques known in the art, such as extraction from a suitable solvent followed removal of solvent from the reaction mixture by evaporation, distillation and the like, any other methods can be employed. Specifically, condensation of compound of formula IIIa (wherein x is p-toluenesulfonyl or methanesulfonyl) with compound of formula IV-2 is accomplished using a base in a suitable solvent. The reaction can optionally be performed in presence of phase transfer catalyst. The reaction can be preferably conducted at a temperature of 10 to 70° C. and it takes about 1-25 hours for the completion of reaction. The compound of formula Va-2 can be isolated from the reaction mixture using any conventional methods. Specifically, the compound of formula Va-2 is isolated from the reaction by the addition of water followed by layer separation using a water immiscible solvent. Preferably water immiscible solvents employed include halogenated solvents such as dichloromethane, chloroform; ethers such as 2-methyl tetrahydrofuran, isopropyl ether, methyl tert-butyl ether; aliphatic or aromatic hydrocarbon such as toluene, xylene, ethyl benzene and the like or mixture thereof. Thereafter, intermediate compound of formula Va-2 is recovered from the solution by any suitable techniques such as removal of solvent using distillation, evaporation and the like.

Similarly, the compound of formula V wherein the R₁, R₂, together, form a double bond provided R₃ and R₄ are hydrogen or R₃, R₄, together, form a double bond provided R₁ and R₂ are hydrogen, has structure formula Vb,

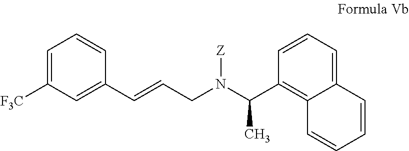

wherein Z is as defined above
may be prepared by the condensation of compound of formula IIIb with compound of formula IV and further forms the inventive part of the invention.

Similarly, the compound of formula V wherein the R₁, R₂, R₃ and R₄ all are combined together to form triple bond, has structure formula Vc,

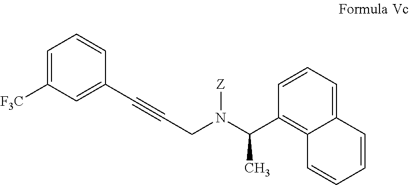

wherein Z is as defined above
may be prepared by the condensation of compound of formula IIIc with compound of formula IV and further forms the inventive part of the invention.

The resulting compound of formula V may be characterized by various spectroscopic techniques like ¹H and ¹³C Nuclear magnetic resonance (NMR), Ultraviolet spectroscopy (UV), Mass spectrometry (MS), Infrared spectroscopy (IR). Further X-ray diffraction pattern of the compound provide information whether compound exist in crystalline or amorphous form. The compound of formula Va-1 exists in solid from, it can occur in different polymorphs or in amorphous form. The crystalline or amorphous nature of the compound is characterized by X-ray diffraction pattern. Further, the compound of formula V, including salts, hydrates, solvates, racemates, enantiomers, polymorphs thereof forms a part of the present invention.

Specifically, the compound of formula Va-1 is characterized by ¹H-NMR (CDCl₃) showing peaks at δ 8.46 (d, 1H); 8.25 (d, 2H); 7.93 (d, 2H); 7.82 (m, 2H); 7.59 (t, 1H); 7.52 (t, 1H); 7.35 (m, 3H); 7.26 (m, 1H); 6.90 (m, 2H); 6.07 (dd, 1H); 3.07 (m, 2H); 2.18 (t, 2H); 1.37 (m, 1H); and 0.8 (m, 1H).

Also compound of formula Va-2 is characterized by ¹H-NMR (CDCl₃) showing peaks at 8.07 (bs, 1H); 7.74 (d, 1H); 7.67 (d, 1H); 7.24-7.44 (m, 4H); 7.19 (d, 1H); 7.08 (t, 1H); 6.80 (bs, 1H); 6.71 (d, 1H); 6.0 (m, 1H); 2.7 (m, 2H); 2.0 (m, 2H); 1.48 (d, 3H); 1.40 (s, 9H); 1.13 (m, 1H); and 0.77 (m, 1H).

The compound of formula V, if desired can be purified to enhance the purity of the desired intermediate or to remove undesired impurities in the intermediate using suitable methods. Any suitable purification procedure such as, for example, crystallization, derivatisation, slurry wash, salt preparation, various chromatographic techniques, solvent, anti-solvent crystallization or combination of these procedures, may be employed to get purified material. However, other equivalent procedures such as acid-base treatment could, also be used, to purify the intermediate of formula V. Solvents used for the purification may be selected depending upon the nature of the compound to be purified, however the solvent can be chosen amongst water, C₁₋₆ alcohols such as methanol, ethanol, tert-butanol, isopropanol; aliphatic C₃₋₆ ketones such as acetone, methyl ethyl ketone, methyl isobutyl ketone; aliphatic or aromatic hydrocarbons such as toluene, xylene, ethyl benzene, n-heptane, cyclohexane, n-hexane; C₃₋₆ ethers such as methyl tertiary butyl ether, isopropyl ether, 2-methyl tetrahydrofuran, dioxane, 1,2-dimethoxy ethane and the like or mixture thereof in suitable proportion.

Specifically, the compound of formula Va can be crystallized using a suitable solvent, in which the compound has some solubility either at room temperature or at higher temperature, that includes C₁₋₆ alcohols such as methanol, ethanol, isopropanol, n-butanol, tert-butanol; aliphatic C₃₋₆ ketones such as acetone, methyl ethyl ketone, methyl isopropyl ketone, methyl isobutyl ketone; aliphatic or aromatic hydrocarbons such as toluene, xylene, ethyl benzene and the like or mixture thereof.

Alternatively, the compound of formula Va can be purified by slurry wash in a suitable solvent, in which the compound has low solubility as compared to impurities, such solvent includes but not limited to aliphatic hydrocarbon such as n-heptane, cyclohexane, hexanes, n-hexane; ether such as methyl tertiary butyl ether, isopropyl ether, 1,2-dimethoxyethane; dioxane, 2-methyl tetrahydrofuran, tetrahydrofuran and the like or mixture thereof in any suitable proportions.

Alternatively, intermediate compound of formula Va can be purified by using a special treatment to remove any specific impurity such as compounds of formulae Vb and Vc, which may be present in the intermediate compound of formula Va. For example, if any impurity having alkene functionality is present in the intermediate compound of formula Va then it can be removed using a suitable reagent that either bind with the alkene functionality to form a complex or change the nature of the alkene impurity so that it can be easily removed or isolate from the reaction mixture by using suitable methods like extraction or filtration and the like. Suitable regent can be selected from oxidizing agent such as potassium permanganate, potassium dichromate, chromic acid; or silver salts such as silver nitrate that bind with the alkene functionality. Preferably, the intermediate compound of formula Va is treated with a suitable reagent for few minutes to several hours. The reaction can optionally be carried out in the presence of inert solvent that includes but not limited to halogenated solvent such as dichloromethane; aliphatic or aromatic hydrocarbon such as toluene; and the like or mixture thereof. Optionally a suitable phase transfer catalyst can be added to the reaction mixture and selected from the list as given described above. Thereafter, purified intermediate free from alkene impurity can be isolated from the reaction mixture by filtration or by the addition of water to make the reaction mixture biphasic. The desired product can be extracted from mixture by the removal of solvent from the organic layer.

In this way, the solvent and type of purification required to enhance the purity of the intermediate can be chosen based on the nature of intermediate of formula V and impurity to be removed. Similarly, compound of formula Vb and Vc can be purified using a suitable methods. The purification processes can be repeated or used in combination with other till the desired purity of the intermediate is achieved.

According to another aspect, present invention provides a process for the conversion of intermediate compound of formula V including salts, hydrates, solvates, racemates, enantiomers, polymorphs thereof to cinacalcet of formula I and its pharmaceutically acceptable salts thereof.

In a preferred embodiment of the present invention, the compound of formula Va, may be converted to cinacalcet and its pharmaceutically acceptable salts thereof.

Generally, the deprotecting agent and the reaction conditions for deprotection of amine protecting group is chosen appropriately depending upon the nature of protecting group. The amino-protecting group can be removed using conventional procedures and reagents. For example, benzyl protecting group or substituted benzyl protecting group can be removed by selective hydrogenolysis in the presence of a catalyst, such as palladium and the like; a tert-butoxycarbonyl group can be removed by treatment with strong acid, such as hydrochloric acid, p-toluenesulfonic acid, trifluoroacetic acid and the like; 9-fluorenylmethyloxycarbonyl can be removed by treatment with a suitable base; a tert-butyldimethylsilyl group can be removed by treatment with a source of fluoride ions, such as triethylamine trihydrofluoride and the like; p-methoxyphenyl can be removed by ammonium cerium (IV) nitrate; p-toluenesulfonyl group can be removed by treatment with concentrated acid such as hydrobromic acid, sulfuric acid and the like or by strong reducing agents such as sodium in liquid ammonia, sodium naphthalene and the like; sulfonamide can be deprotected by substituted or unsubstituted thiophenol; samarium iodide, tributyltin hydride. Appropriate deprotecting agent can be perceived by those well versed in the art from ‘Protecting Groups by Philip J. Kocienski (Thieme, 2000)’ or ‘Protective Groups in Organic Synthesis by Theodora W. Greene, Peter G.M. Wuts’ or available and well documented in the literature. The solvent employed in the reaction can be chosen depending upon the nature of the protecting group to be removed. After the completion of the reaction, cinacalcet can be isolated from the reaction mixture or in situ converted to cinacalcet pharmaceutically acceptable salts. Thus, compound of formula Va, including salts, hydrates, solvates, racemates, enantiomers, polymorphs thereof can be directly converted to cinacalcet pharmaceutically acceptable salts.

In a more preferred embodiment of the present invention, it provides a process for the preparation of cinacalcet and its pharmaceutically acceptable salts thereof by the deprotecting the intermediate compound of formula Va-1.

Typically, the process involves the reaction of compound of formula Va-1 with a suitable deprotecting agent at a temperature 0 to 100° C., till the completion of the reaction. Any deprotecting reagent can be employed in the reaction that can effectively remove p-nitrobenzenesulfonyl group, and selected from any reagent known in the art for such purpose. Preferably, suitable deprotecting agent includes but not limited to substituted or unsubstituted thiophenol, samarium iodide, tributyltin hydride and the like. Preferably, deprotecting agent used is substituted or unsubstituted thiophenol. There is no restriction on the nature of the solvent employed for the reaction, provided it has no adverse effect on other functionality. Particularly, the solvent includes but not limited to ether such as tetrahydrofuran, 2-methyl tetrahydrofuran; amide solvents such as dimethylformamide, dimethylacetamide; aprotic solvent such as dimethylsulfoxide; nitriles such as acetonitrile, propionitrile and the like or mixture thereof in any suitable proportions. The reaction is additionally carried using base with or without phase transfer catalyst. Base employed for the reaction can be organic or inorganic base. Organic base include but not limited to amines such as trialkylamine. Inorganic base includes alkali or alkaline metal hydroxide, carbonate, bicarbonates, hydrides, alkoxide thereof such as potassium carbonate, sodium carbonate, sodium bicarbonate, potassium carbonate and the like. The phase transfer catalyst includes, but not limited to quaternary ammonium compounds: benzyl trimethylammonium chloride and bromide, cetyl trimethylammonium bromide phosphonium compounds or synthetic resins, tetrabutylammonium bromide or chloride; benzyltriethylammonium chloride; tetrabutylammonium hydroxide; tricaprylmethylammonium chloride, dodecyl sulfate, sodium salt, such as sodium lauryl sulfate; tetrabutylammonium hydrogensulfate; hexadecyltributylphosphonium bromide; hexadecyltrimethyl ammonium bromide or resin amberlite IRA-410 and the like. After the completion of reaction, the desired compound i.e. cinacalcet can be isolated from the reaction mixture or reaction mixture is used, as such, for the next step i.e. preparation of cinacalcet pharmaceutically acceptable salts. Cinacalcet can be isolated from the reaction mixture by any conventional method in the art. Specifically, cinacalcet can be isolated from the reaction by removal of solvent, extraction with a suitable solvent, layer separation and the like. Cinacalcet thus obtained or the reaction mixture is made to react with a suitable acid to form cinacalcet pharmaceutically acceptable salts.

In one another more preferred embodiment of the present invention, it provides a process for the preparation of cinacalcet and its pharmaceutically acceptable salts thereof by the deprotecting the intermediate compound of formula Va-2.

Typically, the process involves the reaction of compound of formula Va-2 with a suitable deprotecting agent at a temperature 0 to 100° C. for few minutes to several hours, preferably till the completion of the reaction. Any deprotecting reagent can be employed in the reaction that can effectively remove tert-butyloxycarbonyl group, and selected from any reagent known in the art for such purpose. Preferably, suitable deprotecting agent includes concentrated or aqueous strong acid such hydrochloric acid or trifluoroacetic acid, hydrobromic acid, hydroiodic acid, phosphoric acid, and the like. The acid employed for reaction can be gaseous, aqueous or solvent saturated with acid, mixture of acid with a solvent. There is no restriction on the nature of the solvent used herein but specifically includes ethers such as isopropyl ether-1,2-dimethoxyethane, dioxane, 2-methyl tetrahydrofuran, tetrahydrofuran, methyl tert-butyl ether; alcohols such as methanol, ethanol, propanol, tert-butanol; esters such as ethyl acetate, isobutyl acetate; aliphatic or aromatic hydrocarbons such as toluene; amide solvents such as dimethylformamide; aprotic solvent such as dimethylsulfoxide; and the like or mixture thereof. After the completion of reaction, the desired compound i.e. cinacalcet can be isolated from the reaction mixture or reaction mixture is used as such for the next step, preparation of cinacalcet pharmaceutically acceptable salts. Cinacalcet can be isolated from the reaction mixture by any conventional method in the art. It is advantageous to proceed with the reaction mixture to synthesize cinacalcet hydrochloride.

Alternatively, the compound of formula Va-2 can be converted to cinacalcet hydrochloride without isolation of cinacalcet freebase, by using hydrochloric acid for the deprotection, it directly gives cinacalcet hydrochloride. Hydrochloric acid employed for reaction can be gaseous, aqueous or solvent saturated with hydrogen chloride, mixture of hydrochloric acid with a solvent. There is no restriction on the nature of the solvent used herein but specifically includes ethers such as isopropyl ether-1,2-dimethoxyethane, dioxane, 2-methyl tetrahydrofuran, tetrahydrofuran, methyl tert-butyl ether; alcohols such as methanol, ethanol, propanol, tert-butanol; esters such as ethyl acetate, isobutyl acetate; aliphatic or aromatic hydrocarbons such as toluene; amide solvents such as dimethylformamide; aprotic solvent such as dimethylsulfoxide; and the like or mixture thereof. Cinacalcet hydrochloride, thus prepared, can be isolated by using any conventional techniques.

In another preferred embodiment of the present invention, it provides a process for the preparation of cinacalcet and its pharmaceutically acceptable salts thereof from intermediate compounds of formulae Vb or Vc. The process comprises reducing the compounds of formulae Vb or Vc to form compound of formula Va which is then converted to cinacalcet and its pharmaceutically acceptable salts thereof as described above.

Generally, the compound of formula Vb or Vc is reduced in presence of reducing agent and a suitable solvent at a temperature of 25 to 100° C. to form a compound of formula Va. The reduction reaction can be performed by any methods known in prior art for the complete reduction of double or triple bonded functionality. Preferably, reduction can be carried out by catalytic hydrogenation (hydrogen over a metal catalyst). The metal catalyst includes, but not limited to transition metal, transition metal on support (where support can be carbon or barium sulfate), organometallic compounds of transition metal (homogenous catalyst), or other transition metal derivative or platinum dioxide and the like. The transition metal includes, but not limited to palladium, platinum, rhodium, ruthenium or nickel and the like. The hydrogen pressure employed in the reaction can be from 1 to 5 atmosphere. The hydrogenation is carried till the completion of the reaction, preferably for 1 to 24 hours. Reducing agents include, but not limited to borane complexes such as borane-tetrahydrofuran, borane-dimethylsulfide, borane amine, borane lewis base, borane-triphenylphosphine and the like; hydride transfer reagent. The reducing agents MBR₆H or MAlR₆H can be used with or without cocatalysts, but not limited to cobalt or nickel derivatives and with or without ligands like dimethylglyoxime and the like (wherein M can be metal like alkali metal or alkaline earth metal or transition metal or a suitable metal and R₆ can be any ligand selected from alkoxy, RN, (R)₂N, (R)₃N, RCOO, RS, CN and the like; R is selected from substituted or unsubstituted alkyl, alkenyl, alkynyl, substituted or unsubstituted aryl, substituted or unsubstituted aralkyl and the like); or other appropriate reducing reagent as mentioned in comprehensive organic transformation by R. C. Larock. Suitable solvents includes, C₁₋₅ alcohol, C₅₋₈ aliphatic or aromatic hydrocarbon, C₃₋₈ ester, C₁₋₈ ether, water and the like or mixture thereof. The resulting compound of formula Va is then converted to cinacalcet and its pharmaceutically acceptable salts thereof by the process as described above.

According to another aspect, the present invention provides another process for the preparation of cinacalcet and its pharmaceutically acceptable salts thereof by the reduction of intermediate compound of formula Vc to compound of formula Vb which is then converted to cinacalcet or pharmaceutically acceptable salts thereof by either
a). conversion of compound of formula Vb in to Va which is deprotected to give cinacalcet and its pharmaceutically acceptable salts thereof; or
b). simultaneous reduction of double bond as well as removal of amine protecting group to give cinacalcet and its pharmaceutically acceptable salts thereof.

According to another aspect, the present invention provides a process for the preparation of cinacalcet and pharmaceutically acceptable salts thereof from intermediate compounds of formulae Vb or Vc by the simultaneous reduction of double or triple bond as well as removal of amine protecting group.

Cinacalcet free base can be optionally isolated in the process of present invention. Cinacalcet free base, if isolated can be optionally purified to remove any impurity present in cinacalcet by any suitable methods, specifically by gel purification.

According to one aspect, the present invention provides a process for the purification of cinacalcet by gel purification.

Generally, the process involves the addition of silica gel to the solution of cinacalcet in a suitable solvent. The solution of cinacalcet can be prepared by suspending cinacalcet in a suitable solvent or such a solution can be obtained directly from the reaction mixture in which cinacalcet is formed. Suitable solvents can be selected from but not limited to aromatic or aliphatic hydrocarbon, C₁₋₈ ether, halogenated solvents or mixture thereof. Preferably, the solvent is selected from heptane, cyclohexane, hexane, toluene, o-xylene, m-xylene, p-xylene, isopropyl ether, diethyl ether, methyl tertiary butyl ether, dichloromethane, chloroform or mixture thereof. The solution of cinacalcet in a solvent can be optionally heated at a temperature from about 25 to 135° C. depending upon the solvent used. Any other temperature is also acceptable as long as stability of cinacalcet is not compromised and a clear solution is obtained.

To above solution of cinacalcet, silica gel is added and mixture is stirred for few minutes to few hours, preferably till the complete adsorption takes place on silica gel. Thereafter, solvent is removed by distillation, or evaporation to have complete adsorption of cinacalcet along with impurities on the silica gel. After the removal of solvent, fresh solvent (same as described above) is added to the residue and mixture is again stirred for few minutes to few hours at a temperature of −5 to 35° C., preferably for 30 minutes at room temperature till the complete extraction of cinacalcet from the silica gel. The impurities remain adsorbed on the silica gel. Thereafter, the silica gel is removed from the solution. Silica gel can be removed by suitable technique such as filtration and the like. In order to enhance the yield, product can optionally be extracted from the silica gel by performing one or more extraction with a suitable solvent as described above. Then, cinacalcet is recovered from the filtrate by evaporation of the solvent by distillation. The impurities present in cinacalcet, remain adsorbed on the silica gel, so cinacalcet obtained after purification is free from the polar impurities. Silica gel used for the purification can have mesh size ranges from 50-400 mesh, preferably 230-400 mesh, 100-230 mesh, 200-300 mesh and 50-80 mesh. The ratio of crude cinacalcet to silica gel can be from 1:1 to 1:3, preferably 1:3, more preferably 1:2.

Specifically, cinacalcet is dissolved in a suitable non polar solvent followed by addition of silica gel to the solution. The solvent is removed from the mixture to ensure complete adsorption of cinacalcet along with impurities on silica gel. After complete adsorption suitable non polar solvent is added to extract cinacalcet from silica gel and stirred. Silica gel is removed by the filtration and pure cinacalcet is recovered from the solution by suitable techniques such as distillation.

Cinacalcet obtained before or after the purification can be converted to its pharmaceutical acceptable salts. Preferably, cinacalcet is converted to its pharmaceutically acceptable salts by the processes known in the prior art. Specifically, cinacalcet free base is dissolved in a suitable solvent, such as an aqueous or aqueous-alcohol solution, ester and the like or mixture thereof, containing the appropriate acid and then isolated by evaporating the solution. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Pharmaceutically acceptable salts include acid addition salts such as those containing sulfate, hydrochloride, maleate, phosphate, sulfamate, acetate, citrate, lactate, tartrate, methanesulfonate, ethanesulfonate, benzenesulfonate, p-toluenesulfonate, cyclohexylsulfamate and quinate and others.

Preferably cinacalcet hydrochloride is prepared by treating cinacalcet with hydrochloric acid at 0° C. to 130° C. temperature for few minutes to few hours. The source of hydrogen chloride include but not limited to aqueous hydrochloric acid, hydrogen chloride gas or mixture thereof with suitable solvent selected from alcohol, ester, ether and the like. Preferably, the source of hydrogen chloride includes methanolic hydrochloride, ethyl acetate hydrochloride, and the like. The hydrochloride formation can be carried out in the a solvent selected from ester such as ethyl acetate; ethers such as isopropyl ether, methyl tertiary butyl ether, tetrahydrofuran; nitriles such as acetonitrile; alcohols such as methanol and the like or mixture thereof. Cinacalcet hydrochloride can be isolated from the reaction mixture by the suitable methods such as distillation, evaporation, extraction with a solvent and the like.

The processes of the present invention can be shown by following schemes.

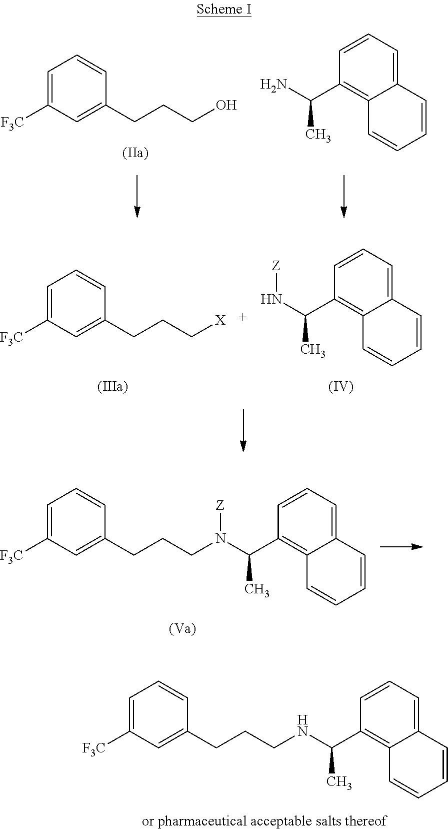

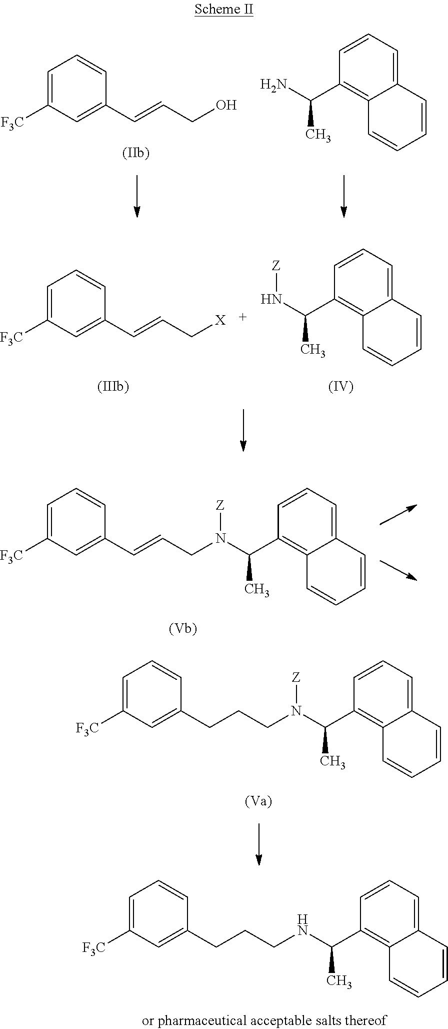

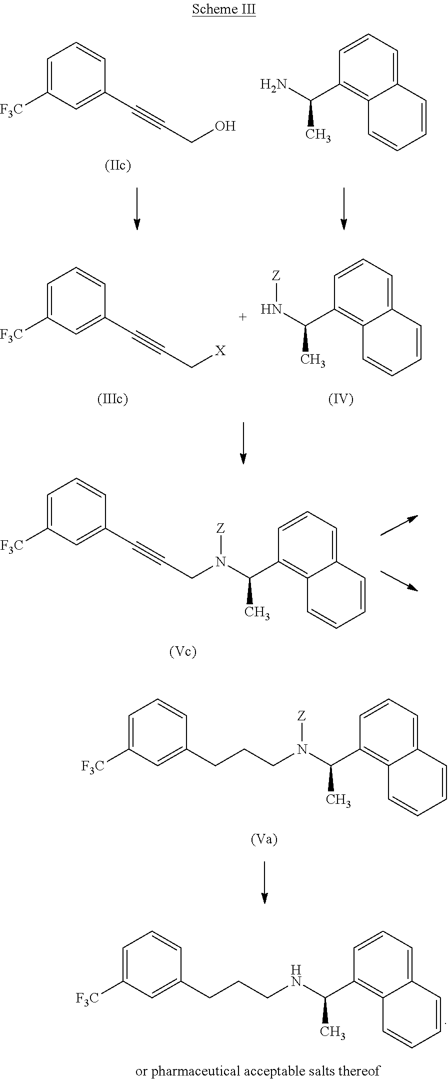

In another embodiment of the present invention, racemic cinacalcet or pharmaceutically acceptable salts thereof can be prepared by the condensation of compound of formula III with racemic compound of formula IV to form racemic compound of formula V which is then converted to racemic cinacalcet and its pharmaceutically acceptable salts thereof by following the same process as described in the present invention. Racemic cinacalcet and pharmaceutically acceptable salts thereof thus prepared can be subjected to a chiral resolution to obtain (R)-cinacalcet and pharmaceutically acceptable salts thereof. The resolution can be performed by treating the racemic compound with a suitable resolving agent in suitable solvents. Resolving agent includes, but not limited to naproxen, tartaric acid, mandelic acid, 2,3:4,6-di-O-isopropylidene-2-keto-gluconic acid and the like. Suitable solvents for the resolution includes, but not limited to C₁₋₅ alcohols; C₃₋₈ ketone; halogenated solvents; C₁₋₆ straight chain, branched, or aromatic chloro hydrocarbons; ethers, preferably the solvents can be selected from acetone, ethyl methyl ketone, diethyl ketone, dichloromethane, dichloroethane, chloroform, carbon tetrachloride, chlorobenzene, dichlorobenzene, tetrahydrofuran, diethylether, methyl tertiary-butyl ether, 1,4-dioxane and the like; and mixtures thereof or their combinations with water in various proportions. The salts of the racemate with enantiomeric acid is separated and then the desired diastereomeric salt is converted to cinacalcet or pharmaceutically acceptable salts thereof.

It is found by the present inventor that condensation of compound of formula IIIa (where X is as defined above) with compound of formula IV (where in Z is selected from hydrogen or a functional group of general formula —COOR″ wherein R″ is as defined above) in presence of a base in organic solvent, may results in the formation of compound of formula Va contaminated with substituted carbamate impurity of formula VII.

If compound of formula Va (contaminated with substituted carbamate impurity of formula VII) is further converted to final product i.e. cinacalcet and its salts thereof, then it is also found to be contaminated with substituted carbamate impurity. It is observed by the present inventors that the number of mole equivalent of compound of formula IIIa used in the reaction is crucial for the impurity generation. With the increase in amount of the compound of formula IIIa, the percentage of impurity in the resulting product increases. It is found that if an excess amount of the compound of formula IIIa is taken for the reaction during reaction the unreacted compound of formula IIIa left in the reaction mixture, reacts with the resulting product and results in the formation of substituted carbamate impurity of cinacalcet in higher amounts.

As is known in the art, the management of process impurities is greatly enhanced by understanding their chemical structures and by identifying the parameters that influence the amount of impurities in the final product, therefore impurity of the present invention can be characterized by various spectroscopic techniques like ¹H and ¹³C Nuclear magnetic resonance (NMR), Ultraviolet spectroscopy (UV), Mass spectrometry (MS), Infrared spectroscopy (IR) to understand its chemical structure. The percentage of impurity present in cinacalcet or its salts can be identified by chromatographic techniques like thin layer chromatography (TLC) or high pressure liquid chromatography (HPLC) preferably, by high pressure liquid chromatography.

The substituted carbamate impurity of the present invention is characterized by mass spectroscopy (“MS”) analysis and found to have molecular weight of 588 g/mole. The mass spectroscopy (“MS”) analysis shows M⁺¹ peak at 589.

The substituted carbamate impurity of the present invention is characterized by following nuclear magnetic resonance (“NMR”) spectral data:

¹H-NMR δ (CDCl₃): 6.83-8.2 (15H, m, Ar—H); 6.23 (1H, bs, CHN); 4.23 (2H, bs, CH₂O); 2.90 (2H, bs, NCH₂); 2.75 (2H, bs, ArCH₂ . . . O); 2.17 (2H, bs, ArCH₂ . . . N); 2.05 (2H, bs, ArCH₂CH₂ . . . O); 1.6 (3H, d, CH₃); 1.3 (2H, bs, ArCH₂CH₂ . . . N).

The substituted carbamate impurity of the present invention is characterized by Infrared (“IR”) spectral data showing the absorption of carbonyl group (C═O) of the impurity at 1693 cm⁻¹.

The identification of the impurities in final products as well intermediates is very important, for this impurity is required, to use as marker. Therefore, present invention provides a process for the preparation of impurity, so that presence or absence of the impurity in a cinacalcet or intermediate of formula Va can be identified or can be checked in any quality control process in order to ensure that the process complies with the required standards set down in the regulatory approval of that product prior to it being released for commercial sale.

According to one embodiment, the present invention provides a process for the preparation of substituted carbamate impurity of cinacalcet, having formula VII.

Generally the reaction involves the condensation of compound of formula IIIa (where X is as defined above) with compound of formula IV (wherein Z is selected from hydrogen or a functional group of general formula —COOR″ wherein R″ is as defined above) in presence of the base and suitable solvent at a temperature of 25° C. to reflux temperature of the solvent for few minutes to 30 hours. Preferably, the reaction mixture is stirred at ambient temperature for 1 to 20 hours. It is advantageous to take excess of compound of formula IIIa for the generation of substituted impurity in high amount. The organic solvent for the reaction includes but not limited to ketones such as methyl isobutyl ketone, methyl ethyl ketone; ethers such as isopropyl ether, methyl tertiary butyl ether; nitriles such as acetonitrile; halogenated solvents such as chloroform, dimethyl sulfoxide; amide solvents such as dimethylformamide; aliphatic or aromatic hydrocarbon such as toluene; and the like or mixture thereof. Base employed in the reaction can be organic or inorganic base. Organic base include amine base such as C₁₋₈ trialkylamine and the like. Inorganic base includes alkali or alkaline metal hydroxide, alkoxide, carbonates, bicarbonates thereof. Preferably, sodium hydroxide is employed in the reaction. The reaction is optionally take place in the presence or absence of phase transfer catalyst. After the completion of the reaction, product can be isolated from the reaction mixture by suitable techniques such as distillation, evaporation, extraction with a suitable solvent and the like. Preferably, the product is isolated by the addition of water followed by extraction with a suitable solvent; thereafter removal of solvent gives product. Suitable extracting solvent include but not limited to aromatic hydrocarbon such as toluene; ether such as isopropyl ether; halogenated solvents such as dichloromethane and the like or mixture thereof.

The product isolated from the reaction mixture is found to be the mixture of compound of formula Va and substituted carbamate impurity of formula VII. The amount of the substituted carbamate impurity of formula VII in the mixture may vary from 1 to 50%, depending upon the amount of starting material, temperature, solvent and other reaction conditions. Preferably the percentage of the substituted carbamate impurity may be present in 5 to 50% by HPLC.

The resulting product i.e. mixture of compound of formula Va and substituted carbamate impurity of formula VII may be further converted to cinacalcet hydrochloride containing substituted carbamate impurity of formula VII as this impurity is carried to final stage.

The cinacalcet carbamate impurity may be separated from the compound of formula Va or from cinacalcet hydrochloride by the techniques known in art for the separation of the impurities from the product. Preferably, it is separated by chromatographic techniques like preparative plate chromatography, column chromatography, flash chromatography and the like. Most preferably, impurity separation is performed by the column chromatography.

Preferably, substituted carbamate impurity of formula VII may be isolated by subjecting the compound of formula Va having substituted carbamate impurity to column chromatography. The column chromatography comprises using a silica gel, as a stationary phase, and a gradient of eluents that remove substituted carbamate impurity of formula VII from the column on which it adsorbed. The stationary phase, a solid adsorbent, is placed in a vertical glass (usually) column and the mobile phase, a liquid is added to the top and flows down through the column (by either gravity or external pressure or vacuum). Column chromatography is generally used as a technique to isolates desired compounds from a mixture. The mixture of compound of formula Va and substituted carbamate impurity is applied to the top of the column. The liquid solvent (the eluent) is passed through the column by gravity or by the application of air pressure or vacuum. Equilibrium is established between the solute adsorbed on the adsorbent and the mobile phase flowing down through the column. Because the different components in the mixture have different interactions with the stationary and mobile phases, they will be carried along with the mobile phase to varying degrees and a separation will be achieved. The substituted carbamate impurity of formula VII is collected as the solvent fraction from the column.

Alternatively, the cinacalcet substituted carbamate impurity of formula VII can be prepared from the cinacalcet-carbamate compound of formula VIII.

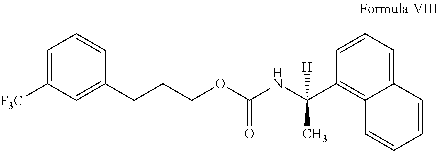

Generally the reaction involves the condensation of cinacalcet carbamate compound of formula VIII with compound of formula IIIa in presence of the base and suitable solvent at a temperature of 0° C. to reflux temperature of the solvent for few minutes to few hours. Preferably, the reaction mixture is stirred at ambient temperature for 2 to 20 hours. The solvent and base employed in the reaction are same as described above for the generation of the impurity. After the completion of the reaction, product can be isolated from the reaction mixture by suitable techniques such as distillation, evaporation, extraction with a suitable solvent and the like. Preferably, the product is isolated by the addition of water followed by extraction with a suitable solvent, thereafter removal of solvent to give product. Suitable extracting solvent include but not limited to aliphatic or aromatic hydrocarbons such as toluene; ethers such as isopropyl ether; halogenated solvents such as dichloromethane and the like or mixture thereof.

The isolated substituted carbamate impurity of formula VII by the process of present invention may have purity around 50% to 92.75% by HPLC. The isolated impurity, if desired, can be further subjected to column chromatography as described above to enhance the purity of the compound. The isolated substituted carbamate impurity of cinacalcet prepared by the processes of present invention or after isolation from the solvent fraction obtained from column is pure. Preferably it has purity not less than 88.0% by HPLC. Preferably, the substituted carbamate impurity is isolated in about 90.0% purity by HPLC; more preferably, the substituted carbamate impurity is isolated in about 92.75% purity by HPLC.

The final product, cinacalcet or cinacalcet hydrochloride thus prepared by the process of present invention can be purified to enhance the purity of the final API or to remove undesired impurities in the intermediate using a conventional methods. Any suitable purification procedure such as, for example, crystallization, derivatisation, slurry wash, salt preparation, various chromatographic techniques, solvent anti-solvent system or combination of these procedures, may be employed to get the purified material. However, other equivalent procedures such as acid-base treatment could, also be used. The solvents used for the purification of final compound and intermediates of the present invention may be selected depending upon the nature of the compound to be purified, however the solvent can be chosen amongst water, C₁₋₆ alcohols such as ethanol, isopropanol; aliphatic C₃₋₆ ketones such as acetone, ethyl methyl ketone, methyl isobutyl ketone; aliphatic or aromatic hydrocarbons such as toluene, n-heptane, cyclohexane; C₃₋₆ ethers such as methyl tertiary butyl ether, isopropyl ether; nitriles such as acetonitrile and the like or mixture thereof in suitable proportion.

Specifically, the present invention provides a process for the removal of substituted carbamate impurity of formula VII from a sample of cinacalcet hydrochloride. The present invention also provides cinacalcet hydrochloride having substituted carbamate impurity less than 0.15%.

Generally, the reaction involves condensation of compound of formula IV with compound of formula IIIa in presence of the base and suitable solvent at a temperature of 25° C. to reflux temperature of the solvent for few minutes to 30 hours. The organic solvent, base and reaction conditions are same as described above for the preparation of compound of formula Va having substituted carbamate impurity of formula VII. The resulting compound of formula Va may contain substituted carbamate impurity in a range of 2 to 20% by HPLC. The compound of formula Va having substituted carbamate impurity is converted to cinacalcet or cinacalcet pharmaceutically acceptable salts. The process involves the reaction of compound of formula Va with a source of hydrogen chloride to form directly cinacalcet hydrochloride. The reaction can be performed with the optional isolation of the cinacalcet free base. The compound of formula Va in a solvent is treated with a source of hydrogen chloride at a temperature of 0 to 130° C. for few minutes to few hours. The compound of formula Va can be optionally in situ converted to cinacalcet hydrochloride. Preferably, the reaction mixture is stirred at ambient temperature for 1 to 6 hours, more preferably till the completion of the reaction. Solvent and source of hydrogen chloride can be selected from the list as described above. Cinacalcet hydrochloride is isolated from the reaction mixture by the suitable techniques such as distillation, evaporation, extraction with a suitable solvent and the like. Preferably, cinacalcet hydrochloride is isolated from the reaction by the removal of solvent. The crude cinacalcet hydrochloride obtained from the reaction mixture is found to contain up to 10% of substituted carbamate impurity of formula VII, preferably up to 5% of substituted carbamate impurity.

Cinacalcet hydrochloride having substituted carbamate impurity is then purified with a suitable solvent to remove the substituted carbamate impurity of formula VII.

The purification process involves the treatment of the cinacalcet hydrochloride with a suitable solvent at a temperature of 0 to 35° C. for few minutes to few hours. Suitable solvent includes but not limited to ester such as ethyl acetate; ethers such as diisopropyl ether, methyl tertiary butyl ether, diethyl ether; hydrocarbon such as n-heptane and the like or mixture thereof in any suitable proportion. Preferably the solvent mixture employed is mixture of ethyl and diisopropyl ether in any suitable proportions. The proportion of the solvents in mixture can vary from 1:1 to 1:100 with respect to cinacalcet hydrochloride, preferably 1:9, more preferably 1:1. Preferably cinacalcet hydrochloride is stirred in suitable solvent at an ambient temperature for 10 minutes to 5 hours, more preferably for 2 hours. The purified product can be isolated from the reaction mixture by the suitable techniques such as filtration and the like. The purification process can be repeated, if desired, to enhance the purity of cinacalcet hydrochloride and to reduce the level of the impurity to the acceptable limit, preferably free from impurity.

In another aspect, the present invention provides cinacalcet hydrochloride having substituted carbamate impurity in amount about 0.03% to 0.15% by HPLC, preferably cinacalcet hydrochloride having substituted carbamate impurity less than 0.15% by HPLC. More preferably, the present invention provides cinacalcet hydrochloride free from the substituted carbamate impurity.

The purity of final product i.e cinacalcet hydrochloride is almost important. Cinacalcet hydrochloride, prepared by the process of present invention or prepared by the methods known in the art needs purification to remove undesired impurities in the product. Therefore, present invention provides processes for the purification of cinacalcet hydrochloride, prepared by any method, to enhance the purity and to minimize identified and unidentified impurities.

According to one aspect, the present invention provides a method for the purification of cinacalcet hydrochloride by employing slurry wash with suitable solvent or solvent mixture.

Generally, the process involves the stirring of slurry of cinacalcet hydrochloride in a suitable solvent at a temperature of −10 to 70° C. for 1 to 5 hours. Preferably, cinacalcet hydrochloride is slurried with suitable solvents at a temperature of 25 to 50° C. for 1 hour. Suitable solvents include but not limited to ester such as ethyl acetate; ethers such as diisopropyl ether, methyl tertiary butyl ether, diethyl ether, hydrocarbon solvents such as n-heptane and the like or mixture thereof in any suitable proportion. The proportion of the solvents in mixture can vary from 1:1 to 1:100, preferably 1:9, more preferably 1:1. It is advantageous to employ the slurry wash with 1:9 mixtures of solvents followed by 1:1 mixture of solvents. Preferably, a mixture of ethyl acetate and diisopropyl ether is employed. Cinacalcet hydrochloride is isolated from the mixture by the suitable techniques such as filtration, and the like. Process of purification can be repeated to enhance the purity of cinacalcet hydrochloride and reduce the impurities level in cinacalcet hydrochloride.

According to another aspect, the present invention provides a method for the purification of cinacalcet hydrochloride by washing with water or aqueous solution of a suitable acid. Generally, the process involves the dissolution of cinacalcet hydrochloride in a suitable solvent followed by washing with water or aqueous solution of a suitable acid followed by water at a temperature of −10 to 70° C. for few minutes to 7 hours, preferably at a temperature of 40 to 50° C. for 0.5 hours. Suitable solvent include aromatic solvent such as toluene; ester such as ethyl acetate; halogenated solvent such as dichloromethane, chloroform; and the like or mixture thereof. Suitable acid is selected from inorganic acid such as hydrochloric acid. After washing process, the aqueous layer is separated out. The solvent is removed from the organic layer by the suitable techniques such as evaporation, distillation and the like. Cinacalcet hydrochloride is isolated from the reaction mixture by any suitable methods. Preferably, the isolation of cinacalcet hydrochloride can be carried out by the addition of suitable solvent to the resulting residue at a temperature of 0 to 40° C. followed by stirring for few minutes to few hours. Preferably, the mixture is slurried at ambient temperature for 45 minutes. Suitable solvent include but not limited to ester such as ethyl acetate; ethers such as diisopropyl ether, methyl tertiary butyl ether, diethyl ether; hydrocarbon solvents such as n-heptane and the like or mixture thereof in any suitable proportion. The proportion of the solvents in mixture can vary from 1:1 to 1:100, preferably 1:9, more preferably 1:1. It is advantageous to employ the slurry wash with 1:9 mixtures of solvents followed by 1:1 mixture of solvents. Mixture of ethyl acetate and diisopropyl ether is preferably employed. Above process of purification remove the unidentified and identified impurities from cinacalcet hydrochloride.

According to another embodiment, the present invention provides a process for purification of cinacalcet hydrochloride by neutralization of cinacalcet hydrochloride to cinacalcet followed by treatment with lithium aluminium hydride and then further conversion to highly pure cinacalcet hydrochloride.

Generally, the process involves the addition of suitable base to a solution of cinacalcet hydrochloride at a temperature of −20° C. to 40° C. for few minutes to few hours. Preferably, the reaction mixture is stirred at ambient temperature for 1 to 5 hours. The solution of cinacalcet hydrochloride can be prepared by mixing cinacalcet hydrochloride in a suitable solvent or such a solution can be obtained directly from a reaction mixture in which cinacalcet hydrochloride is formed. Suitable solvents can be selected from but not limited to aliphatic or aromatic hydrocarbon such toluene, xylene, n-hexane, cyclohexane, n-heptane; ether such as isopropyl ether, diethyl ether, methyl tertiary butyl ether, tetrahydrofuran, 2-methyl tetrahydrofuran; ester such as ethyl acetate; halogenated solvents such as dichloromethane, chloroform and the like or mixture thereof. Suitable base can be organic or inorganic. Organic base include trialkylamine such as triethylamine and the like. Inorganic base include alkali or alkaline metal hydroxide, carbonate, bicarbonate, alkoxide or hydrides thereof. Preferably, the inorganic base is selected sodium hydroxide, sodium carbonate or sodium methoxide. More preferably sodium carbonate is employed in the reaction. After the completion of the reaction, the solvent is removed from the reaction mixture by suitable techniques such as evaporation, distillation and the like. It is optional to isolate cinacalcet from the reaction mixture; residue obtained after solvent removal can be used as such for the further reaction.

The resulting residue in a suitable solvent is stirred with lithium aluminium hydride for few minutes to few hours at a temperature of −5 to 5° C., preferably stirred for 0.5 to 2 hours at 0° C. temperature. Suitable solvents include ether such as tetrahydrofuran, 2-methyl tetrahydrofuran, dioxane, 1,2-dimethoxy ethane, diethyl ether, isopropyl ether, methyl tertiary butyl ether; aliphatic or aromatic hydrocarbon such as toluene, xylene; and the like or mixture thereof. The solution of cinacalcet in a solvent can be optionally cooled at a temperature of below 0° C. followed by addition of lithium aluminum hydride. The reaction can be quenched by the addition of suitable quenching agent such as ester (ethyl acetate), alcohol (methanol) or mixture thereof. After quenching; solvents are distilled off to obtain a residue. Above residue is treated with a source of hydrogen chloride in presence of solvent to give highly pure cinacalcet hydrochloride. The source of hydrogen chloride include but not limited to hydrochloric acid, hydrogen chloride gas or mixture thereof with suitable solvent selected from water, alcohol, ester, aromatic hydrocarbon, ether and the like. Preferably, the source of hydrogen chloride includes methanolic hydrochloride, ethyl acetate hydrochloride, toluene hydrochloride, aqueous hydrochloric acid and the like. The solvent for the preparation of hydrochloride can be selected from ether such as isopropylether, methyl tertiary butyl ether, diethyl ether and the like or mixture thereof. Cinacalcet hydrochloride is isolated from the reaction mixture by the suitable techniques such as distillation, evaporation, extraction with a suitable solvent and the like. Preferably, cinacalcet hydrochloride is isolated from the reaction by the removal of solvent. Cinacalcet hydrochloride obtained by the processes of present invention has high degree of chemical purity and optical purity, according to HPLC (high pressure liquid chromatography). In one aspect, the invention provides, cinacalcet hydrochloride having purity not less than 97% area by HPLC, preferably not less than 99% area by HPLC, and more preferably not less than 99.5% area by HPLC and contains total impurities like identified and unidentified in the amount of less than about 0.5%, or individual impurities less than about 0.15% by weight, more preferably free from the impurities. Starting compounds of formula II can be prepared by the methods known in the prior art such as the methods reported in EP 0194764 A1 or the processes of the present invention described here. According to another aspect, the present invention provides a novel process for the preparation of compound of formula II by the reduction of compound of formula VI,

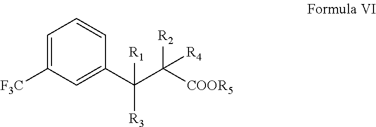

wherein R₁, R₂, R₃ and R₄ are as defined above and R₅ can be selected from hydrogen, alkyl such as methyl, ethyl and the like or any suitable activating group.
in presence of a reducing agent.

In one variant of the compound of formula VI, the R₁, R₂, R₃, and R₄, all are hydrogen and thus compound of formula VI has the structure VIa

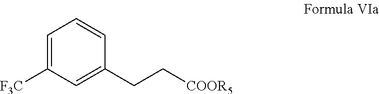

wherein R₅ is as defined above.

In another variant of the compound of formula VI, the R₁ and R₂, together, form a double bond provided R₃ and R₄ are hydrogen or R₃, R₄, together, form a double bond provided R₁ and R₂ are hydrogen and thus compound of formula VI has the structure VIb,

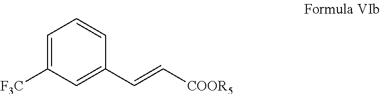

wherein R₅ is as defined above.

In one variant of the compound of formula VI, the R₁, R₂, R₃, and R₄, all together, form a triple bond and thus compound of formula VI has the structure VIc,

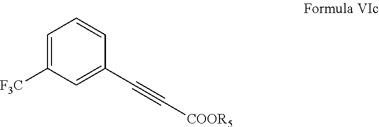

wherein R₅ is as defined above.

According to one aspect of the invention, the present invention provides a process for the preparation of compound of formula IIa by the reduction of compound of VI.

Specifically, the compound of formula VI can be reduced to compound of formula IIa. The reduction can be performed by catalytic hydrogenation (hydrogen over a metal catalyst). The metal catalyst includes, but not limited to transition metal, transition metal on support (where support can be carbon or barium sulfate), organometallic compounds of transition metal (homogenous catalyst), or other transition metal derivative or platinum dioxide and the like. The transition metal includes, but not limited to palladium, platinum, rhodium, ruthenium or nickel and the like. The hydrogen pressure employed in the reaction can be from 1 to 5 atmospheres. The hydrogenation is carried till the completion of the reaction, preferably for 1 to 24 hours. Reducing agents include, but not limited to borane complexes such as borane-tetrahydrofuran, borane-dimethyl sulfide, borane amine, borane lewis base, borane-triphenylphosphine and the like; hydride transfer reagent. The reducing agents MBR₆H or MAlR₆H (wherein M and R₆ are as defined above) can be used with or without cocatalysts include, but not limited to cobalt or nickel derivatives and with or without ligands like dimethylglyoxime and the like.

or other appropriate reducing reagent as mentioned in comprehensive organic transformation by Richard C. Larock. The suitable solvent for the reduction reaction can be selected depending upon the reaction conditions and nature of reducing agent. Suitable solvents includes, but not limited to C₁₋₅ alcohol, C₅₋₈ aliphatic or aromatic hydrocarbon, C₃₋₈ ester, C₂₋₈ ether, water and the like or mixture thereof.

According to another aspect, the present invention provides a novel process for the preparation of compound of formula IIb by the reduction of compound of VI.

Specifically, the compound of formula IIb is prepared by the reduction of compound of formula VIc or alternatively, by the selective reduction of compound of formula VIb. The reduction reaction can be performed by using reducing agents and solvent as described above for the reduction.

According to another aspect, the present invention provides a novel process for the preparation of compound of formula IIc by the reduction of compound of VIc.

According to another aspect, the present invention provides another process for the preparation of compound of formula IIa or IIb.

The compound of formula IIa can be prepared by the reduction of compound of formula IIb or IIc with a suitable reducing agent. Similarly, the compound of formula IIb can be prepared by the selective reduction of compound of formula IIc with a suitable reducing agent. Reducing agent can be selected from as mentioned in comprehensive organic transformation by Richard C. Larock or as mentioned above.

Similarly, starting compounds of formula IV can be prepared by the methods known in the prior art. Specifically, the starting compound of formula IV can be prepared by the introduction of amine protecting group in 1-naphthalen-1-yl-ethylamine and isomers thereof. The protecting group on the 1-naphthalen-1-yl-ethylamine and isomers thereof can be introduced using any appropriate reagent suitable for the protection and condensation and which can be removed at the later stage using appropriate deprotecting agent. Appropriate reagent and appropriate deprotecting agent for amine can be perceived by those well versed in the art from ‘Protecting Groups by Philip T. Kocienski (Thieme, 2000)’ or ‘Protective Groups in Organic Synthesis by Theodora W. Greene, Peter G. M. Wuts’ or available and well documented in the literature. The suitable protecting group includes, but not limited to aromatic or aliphatic sulfanyl halide, aryl, substituted aryl, alkoxy carbonyl, substituted alkoxy carbonyl, aryloxy carbonyl, substituted aryloxy carbonyl, silicon derivatives and the like where substituent can be selected from halogen, alkyl and the like. Preferably protecting group is selected amongst carbobenzyloxy, p-methoxybenzyl carbonyl, tert-butyloxycarbonyl, 9-fluorenylmethyl oxycarbonyl, benzyl, p-methoxybenzyl, 3,4-dimethoxybenzyl, benzyloxycarbonyl group, p-methoxyphenyl, tert-butyldimethylsilyl; other sulfonyl such as p-nitrobenezenesulfonyl, methanesulfonyl, p-toluenesulfonyl, benzenesulfonyl group, and the like. Suitable solvent includes, but not limited to halogenated hydrocarbon, C₃₋₈ ketone, C₅₋₈ aliphatic or aromatic hydrocarbon, C₃₋₈ ester, C₂₋₈ ether, water, C₂₋₅ nitriles, dimethylformamide, dimethylacetamide, dimethyl sulfoxide, N-methylpyrrolidone and the like or mixture thereof. Suitable bases can be selected from an organic or an inorganic base. Organic bases includes, but not limited to tertiary amines; RM or RMgX (wherein R can be alkyl or aryl and M can be alkali or alkaline earth metal); or alkoxide of alkali or alkaline earth metal. Inorganic bases, includes but not limited to alkali or alkaline earth metal hydride, or hydroxide or carbonate or bicarbonate; or MNH₂ or MNSiR₇ (wherein M and R₇ are as defined above); or organometallic bases with or without additives. Optionally, a phase transfer catalyst can be added to the reaction mixture. Phase transfer catalyst can be selected from the list as described above. The phase transfer catalyst may be present in an amount of about 0.05 to about 1.0 mol, preferably 0.05 to 0.5 mol equivalents.

Specifically, the compound of formula IV-1 can be prepared by the reaction of 1-naphthalen-1-yl-ethylamine or isomers thereof with suitable reagent containing p-nitrobenzene sulfonyl group using a suitable base with or without phase transfer catalyst at a temperature sufficient for the completion of the reaction. The suitable reagent can be selected amongst p-nitrobenzenesulfonyl haides, anhydride or mixed anhydride thereof, preferably reaction is carried out using p-nitrobenzenesulfonyl halide. The reaction is generally carried for few minutes to several hours, preferably for 5 hours, more preferably till the completion of the reaction. The reaction is carried out in the presence of the solvent that includes water, halogenated solvent such as dichloromethane, chloroform; ethers such as tetrahydrofuran, 2-methyl tetrahydrofuran, isopropyl ether; toluene, acetonitrile and the like or mixture thereof. Base and phase transfer catalyst employed for the reaction are as described above.

Also specifically, the compound of formula IV-2 can be prepared by the reaction of 1-naphthalen-1-yl-ethylamine or isomers thereof with a suitable reagent containing tert-butyloxycarbonyl group using a suitable base with or without phase transfer catalyst at a temperature sufficient for the completion of the reaction. The suitable reagent can be selected amongst ditertiarybutyl dicarbonate and any other that is capable of introducing tert-butyloxycarbonyl group and the like. The reaction is generally carried for few minutes to several hours, preferably till the completion of the reaction. The reaction is preferably carried out in the presence of the solvent that includes water, ether solvent such as tetrahydrofuran, 2-methyl tetrahydrofuran; ethers such as isopropyl ether, methyl test-butyl ether; halogenated solvents such as dichloromethane, chloroform and the like or mixture thereof. Base and phase transfer catalyst employed for the reaction are as described above.

The intermediates of the present invention can be isolated or used as such in the next step without isolation or optionally recovered from the reaction mixture by suitable techniques known in prior art such as evaporation, filtration or washing and the like. Isolation and purification of final compound and intermediates described here in the present invention can be effected, if desired, by any suitable separation or purification procedure such as, for example, filtration, extraction, crystallization, derivatization, slurry wash, salt preparation or combination of these procedures. However, other equivalent procedures such as acid-base treatment could, of course, also be used. Preferably, intermediates are used directly in the next stage without any purification.

The order and manner of combining the reactants at any stage of the process are not important and may be varied. The reactants may be added to the reaction mixture as solids, or may be dissolved individually and combined as solutions. Further, any of the reactants may be dissolved together as sub-groups, and those solutions may be combined in any order. Wherever required, progress of the reaction is monitored by suitable chromatographic techniques such as High performance liquid chromatography (HPLC) or thin layer chromatography (TLC).

As used, herein the term “conventional methods” may be varied depending upon the nature of the reactions, nature product of the reaction, medium of the reaction and the like the suitable conventional methods can be selected amongst but not limited to distillation of the solvent, addition of water to the reaction mixture followed by extraction with water immiscible solvents, removal of the insoluble particles from the reaction mixture, if present, by filtration or centrifugation or by decantation, addition of water immiscible organic solvent, addition of a solvent to the reaction mixture which precipitate the product, neutralizing the reaction mixture with a suitable acid or base whichever is applicable.

The intermediate described here in the present invention include their salts, hydrates, solvates, racemates, enantiomers, polymorphs and the like.

The major advantage of the present invention is to provide a novel, efficient and industrially advantageous process for preparation of cinacalcet and its pharmaceutically acceptable salts thereof. Further, the present invention also provides novel nitrogen protected intermediates that can be efficiently used in the commercial synthesis of cinacalcet and its pharmaceutically acceptable salts thereof. The another advantages of the present invention lie in isolation of substituted carbamate impurity of the cinacalcet hydrochloride and to provide cinacalcet hydrochloride having substituted carbamate impurity less than 0.15% or preferably free from the substituted carbamate impurity of formula I.

Although, the following examples illustrate the present invention in more detail, but should not be construed as limiting the scope of the invention.

### Example 1

### Preparation of 3-(3-trifluoromethyl-phenyl)-propan-1-ol

Method A: To a solution of 3-(3-trifluoromethyl-phenyl)-propionic acid (5 g, 0.023 mol) in tetrahydrofuran (25 ml) was added borane-dimethylsufide (1.74 g, 0.023 mol) and refluxed for 2 hours. Reaction mixture was cooled and methanol (10 ml) was added at 5-10° C. Solvents were distilled off followed by addition of isopropylether (25 ml) and 5N hydrochloric acid (20 ml). The reaction mixture was heated at 45-50° C. for 2 hours and then cooled to 25-30° C. The layers were separated; organic layer was washed with water, dried and evaporated to give 4.11 g of the title compound.

Method B: A solution of 3-(3-trifluoromethyl-phenyl)-propionic acid (300 g, 1.375 mol) in toluene (1.5 L) was azeotroped for 1 hour and cooled to 40-45° C. Thereafter, borane-dimethylsufide (126.52 g, 1.67 mol) was added and reaction mixture was heated for 3-4 hours at 85° C. and cooled to 0-5° C. The reaction mixture was quenched with methanol (900 ml) and stirred at 0-5° C. for 1 hour. Solvent was distilled off under vacuum 50-55° C. The resulting residue was dissolved in toluene (900 ml) and washed with water (600 ml). Toluene was distilled off under vacuum at 60-65° C. to give 274.38 g of the title compound having purity 97% by HPLC.

### Example 2

### Preparation of toluene-4-sulfonic acid 3-(3-trifluoromethyl-phenyl)-propyl ester

p-Toluenesulfonyl chloride (11.21 g, 0.0588 mol) was added to a solution of 3-(3-trifluoromethyl-phenyl)-propan-1-ol (10 g, 0.049 mol), triethylamine (9.0 ml, 0.06468 mol), 4-N,N-dimethylaminopyridine (0.66 g, 0.0054 mol) in dichloromethane (50 ml) at 25-30° C. The reaction mixture was stirred at a temperature of 35-40° C. for 15 hours. Thereafter, the layers were separated and dichloromethane layer was washed with water (2×20 ml) and dried over anhydrous sodium sulphate. Solvent was distilled off to give 15 g of title compound.

### Example 3

### Preparation of toluene-4-sulfonic acid 3-(3-trifluoromethyl-phenyl)-propyl ester

To a stirred solution of p-toluenesulfonyl chloride (360 g, 1.89 mol) in dichloromethane (1.0 L), triethylamine (243 g, 2.4 mol) and 4-N,N-dimethyl amino pyridine (21 g, 0.17 mol) was added. Thereafter, the reaction mixture was cooled to 0° to −5° C. followed by addition of 3-(3-trifluoromethyl-phenyl)-propan-1-ol (350 g, 1.714 mol) and maintained at 0 to 10° C. temperature for 3 hours. Water (1.05 L) was added to the reaction mixture and stirred for 15 minutes. Layers were separated and washed sequentially with sodium carbonate (1.05 L, 10%), hydrochloric acid (1.05 L, 1N) and water (1.05 L). The organic layer was dried over anhydrous sodium sulphate and was distilled off under vacuum at 25-30° C. to give 522 g of title compound having purity 97.67% by HPLC.

### Example 4

### Preparation of methanesulfonic acid 3-(3-trifluoromethyl-phenyl)-propyl ester

To a stirred solution of 3-(3-trifluoromethyl-phenyl)-propan-1-ol (250 g, 1.224 mol) and triethylamine (148.52 g, 1.47 mol) in dichloromethane (1.25 L), methanesulfonyl chloride (161.32 g, 1.41 mol) was added at 25° C. to 40° C. and reaction mixture was stirred for 2-3 hours at 40° C. Thereafter, reaction mixture was washed with demineralized water (500 ml×3) and dried over anhydrous sodium sulfate. The dichloromethane was distilled off to give 335 g of title compound having purity 91.58% by HPLC.

### Example 5

### Preparation of (R)-(1-naphthalen-1-yl-ethyl)-carbamic acid tert-butyl ester

Method A: (R)-1-Naphthalen-1-yl-ethylamine (5.0 g, 0.0292 mol) was added to a mixture of di-tertiarybutyl dicarbonate (10.0 g, 0.04582 mol) in water (25 ml) and tetrahydrofuran (0.5 ml) at 25-30° C. and stirred for 5 hours. Reaction mixture was then extracted with dichloromethane (3×15 ml). The combined extracts were washed with water and the solvent was distilled off to give 8.2 g of title compound having purity 99.2% by HPLC.

Method B: To a solution of (R)-1-naphthalen-1-yl-ethylamine (150 g, 0.876 mol) in dichloromethane (750 ml), di-tertiarybutyl dicarbonate (210.3 g, 0.964 mol) was added at ambient temperature and stirred for 2 hours. Dichloromethane was distilled off followed by addition of n-heptane (150 ml) and then n-heptane was distilled off. Again n-heptane (900 ml) was added to the resulting residue and stirred. The resulting product was filtered and dried in vacuum at 45-50° C. to give 223 g of the title compound having purity 99.8% by HPLC.

### Example 6

### Preparation of (R)-(1-naphthalen-1-yl-ethyl)-[3-(3-trifluoromethyl-phenyl)-propyl]-carbamic acid tert-butyl ester

Method A: (R)-(1-Naphthalen-1-yl-ethyl)-carbamic acid tert-butyl ester (1.5 g, 5.528 mmol.) was added to a mixture of sodium hydride (50% dispersion in mineral oil, 0.402 g, 0.0084 mol) and dimethylsulfoxide (10 ml) and stirred for 30 minutes at 50-55° C. followed by addition of a solution of toluene-4-sulfonic acid 3-(3-trifluoromethyl-phenyl)-propyl ester (2.0 g, 5.58 mmol) in dimethylsulfoxide (1 ml). The reaction mixture was heated at 50-55° C. for 1 hour. Thereafter, the reaction mixture was cooled to 5° C., quenched with ice-water (20 ml), extracted with isopropyl ether (3×25 ml). Combined isopropyl ether layer was washed with water (2×20 ml) and dried over anhydrous sodium sulphate. Solvent was distilled off to give 1.9 g of title compound.

Method B: Methanesulfonic acid 3-(3-trifluoromethyl-phenyl)-propyl ester (7.28 g, 1.4 meq), (R)-(1-naphthalen-1-yl-ethyl)-carbamic acid tert-butyl ester (5.0 g) and sodium hydroxide (2.95 g) were taken in dimethyl sulfoxide (25 ml) and reaction mixture was stirred at 25-30° C. for 20 hours. Water (50 ml) was added to the reaction mixture and extracted with toluene. Toluene layer was separated and distilled off to give 8.4 g of title compound having substituted carbamate impurity 11.62% by HPLC.

Method C: (R)-(1-Naphthalen-1-yl-ethyl)-carbamic acid tert-butyl ester (210 g, 0.774 mol) was added to a stirred suspension of sodium hydroxide (124 g, 3.1 mol) in dimethylsulfoxide (1.05 L) at 15-20° C. and stirred for 30 minutes. Thereafter, methanesulfonicacid-3-(3-trifluoromethyl-phenyl)-propyl ester (284 g, 1.006 mol) was added to reaction mixture at 20-25° C. and stirred for 20 hours at 25-30° C. The reaction mixture was cooled to 10-15° C. followed by addition of chilled water (2.1 L). The reaction mixture was extracted with toluene (1.0 L×2) and combined toluene extracts were washed with brine (420 ml×1). Then solvent was distilled off under vacuum at 50-60° C. to give 374 g of the title compound having purity 83.59% by HPLC.

Method D: Methanesulfonic acid 3-(3-trifluoromethyl-phenyl)-propyl ester (284 g, 1.3 meq), (R)-(1-naphthalen-1-yl-ethyl)-carbamic acid tert-butyl ester (210 g) and sodium hydroxide (124 g) were taken in dimethyl sulfoxide (1.05 L) and reaction mixture was stirred at 25-30° C. for 20 hours. Water (2.1 L) was added to the reaction mixture and the reaction mixture was extracted with toluene. Toluene layer was separated and distilled off to give 350 g of title compound having substituted carbamate impurity 5.14% by HPLC.

### Example 7

### Preparation of Cinacalcet Hydrochloride

Method A: (R)-(1-Naphthalen-1-yl-ethyl)-[3-(3-trifluoromethyl-phenyl)-propyl]-carbamic acid tert-butyl ester (1 g) was added to 5N hydrochloric acid (15 ml) and reaction mixture was heated at 80-85° C. for 6 hours and then at 25-30° C. for 1 hour. Isopropyl ether (5 ml) was added to the reaction mixture and stirred for 5 minutes. The reaction mixture was filtered, washed with water (2 ml), then with isopropyl ether (2 ml) and dried to give 0.4 g of title compound having purity 99.52% by HPLC.

Method B: To a solution of (R)-(1-naphthalen-1-yl-ethyl)-[3-(3-trifluoromethyl-phenyl)-propyl]-carbamic acid tert-butyl ester (10 g, having substituted carbamate impurity 7.8% by HPLC) in toluene (50 ml) was added concentrated hydrochloric acid (30%, 50 ml) and refluxed for 2 hours. The reaction mixture was washed with water (1.05 L×3) at 40° C. The solvent was distilled off to give 8.0 g of title compound having purity 86.46% by HPLC, and substituted carbamate impurity 5.7% by HPLC.

Method C: To (R)-(1-naphthalen-1-yl-ethyl)-[3-(3-trifluoromethyl-phenyl)-propyl]-carbamic acid tert-butyl ester (350 g, having substituted carbamate impurity 5.14% by HPLC) was added ethyl acetate-hydrochloride (12%, 2.33 L) and stirred for 4 hours. The reaction mixture was washed with water (1.05 L×3) at 40° C. The solvent was distilled off to give 280 g of title compound having purity 87.24% by HPLC, and substituted carbamate impurity 2.82% by HPLC.

Method D: To (R)-(1-naphthalen-1-yl-ethyl)-[3-(3-trifluoromethyl-phenyl)-propyl]-carbamic acid tert-butyl ester (374 g, 0.817 moles) was added ethyl acetate-HCl (12%, 2.33 L) at 20-25° C. in a duration of 2-4 hours. The reaction mixture was then cooled to 0-5° C. followed by washing with chilled water (1.1 L×3). The organic layer was dried over anhydrous sodium sulphate followed by removal of ethyl acetate by distillation under vacuum (150 mm/Hg) at 50-55° C. The resulting product was stirred in ethyl acetate:Isopropyl ether (1:9, 1.75 L) for 1 hour filtered and dried under vacuum at 50° C. to give 227 g of the title compound having purity 99.81% by HPLC.

### Example 8

### Preparation of (R)—N-(1-naphthalen-1-yl-ethyl)-4-nitro-benzenesulfonamide

Method A: To a stirred solution of (R)-1-napthalen-1-yl-ethylamine (100 g, 0.583 mol) triethylamine (97.64 ml, 0.701 mol) and 4-N,N-dimethyl amino pyridine (7.13 g 10 mol %) in dichloromethane (500 ml), p-nitrobenzene sulphonyl chloride (129.42 g, 0.584 mol) was added at 25-30° C. and stirred 6-8 hours. The reaction mixture was washed successively with aqueous hydrochloric acid (150 ml×2), demineralized water (300 ml×2) and dried over anhydrous sodium sulphate. Dichloromethane was distilled off to give 200 g of title compound, which was further dissolved in isopropanol (1.0 L) at 80-85° C., stirred at room temperature for 3 hours, filtered and dried under vacuum to give 167 g of title product having purity 85.40% by HPLC.

Method B: A solution of (R)-1-napthalen-1-yl-ethylamine (200 g, 1.17 mol) in dichloromethane (1.5 L) was added to a stirred solution of sodium carbonate (371.4 g, 3.5 mol), water (2.0 L) and triethylbenzylammonium chloride (26.6 g, 0.117 mol) at 25° to 35° C. Thereafter, p-nitrobenzene sulfonyl chloride (310.6 g, 1.40 mol) and dichloromethane (500 ml) were added to the reaction mixture followed by stirring at 38° to 40° C. for 4 hours. Layers were separated and successively washed with sodium carbonate (1.0 L, 10%), 5N hydrochloric acid (1.0 L) and water (1.0 L). The organic layer was distilled off. To the resulting residue, n-heptane (1.6 L) was added, stirred, filtered and dried under vacuum to give 413 g of the title product having purity 88.14% by HPLC.

### Example 9

### Purification of (R)—N-(1-naphthalen-1-yl-ethyl)-4-nitro-benzenesulfonamide

(R)—N-(1-naphthalen-1-yl-ethyl)-4-nitro-benzenesulfonamide (400 g, having purity 88.14% by HPLC) was dissolved in ethanol (800 ml) at 85-90° C., and stirred at 25° to 30° C. for 3 hours. The resulting product was filtered and dried under vacuum to give 330 g of pure title compound having purity 89.14% by HPLC.

### Example 10

### Preparation of (R)-4-nitro-N-(1-naphthalen-1-yl-ethyl)-N-[3-(3-trifluoromethyl-phenyl)-propyl]-benzene sulphonamide

Method A: (R)—N-(1-Naphthalen-1-yl-ethyl)-4-nitro-benzenesulfonamide (5 g,) was added to a stirred suspension of potassium carbonate (5.8 g), and toluene (50 ml). The reaction mixture was stirred for 1 hour at ambient temperature. Thereafter, toluene-4-sulfonic acid 3-(3-trifluoromethyl-phenyl)-propyl ester (10.05 g) was added to the reaction mixture and heated at 85-90° C. for 10-15 hours. The reaction mixture was then cooled to 25° C., washed with demineralized water (30 ml×2) and dried over sodium sulphate. The solvent was distilled off and n-heptane (50 ml) was added to the resulting residue. The reaction mixture was stirred for 2 hours, filtered and dried under vacuum to give 6.5 g of title compound having purity 93% by HPLC.

Method B: (R)—N-(1-Naphthalen-1-yl-ethyl)-4-nitro-benzenesulfonamide (10.0 g, 0.028 mol) was added to a stirred suspension of potassium carbonate (11.62 g, 0.08 mol), triethylbenzylammonium chloride (0.63 g, 10 mol %) and methyl isobutyl ketone (100 ml) and stirred for 1 hour at ambient temperature. Thereafter, toluene-4-sulfonic acid 3-(3-trifluoromethyl-phenyl)-propyl ester (15.1 g, 0.042 mol) was added to the reaction mixture and heated at 85-90° C. for 15 hours. The reaction mixture was then cooled to 25° C., washed with demineralized water (30 ml×2), aqueous hydrochloric acid (30 ml, 1%) and dried over sodium sulphate. The solvent was distilled off and n-heptane (50 ml) was added to the resulting residue. The reaction mixture was stirred for 2 hours, filtered under vacuum and dried under vacuum to give 14.5 g of title compound having purity 95% by HPLC.

Method C: (R)—N-(1-Naphthalen-1-yl-ethyl)-4-nitro-benzenesulfonamide (320 g, 0.9 mol) was added to a stirred suspension of potassium carbonate (434.15 g, 3.14 mol), triethylbenzylammonium chloride (20.47 g, 10 mol %) and toluene (2.5 L) and stirred for 1 hour at 25-30° C. Thereafter, toluene-4-sulfonic acid 3-(3-trifluoromethyl-phenyl)-propyl ester (483 g, 1.35 mol) in toluene (700 ml) was added to the reaction mixture and heated at 65-70° C. for 15 hours. The reaction mixture was then cooled to 25° C. Water (3.2 L) was added to the reaction mixture, stirred and filtered. The organic layer was separated and washed sequentially with water (3.2 L), hydrochloric acid (1%, 960 ml) and again water (960 ml×2). The solvent was distilled off under vacuum (150 mm/Hg) at 65-70° C. To the resulting residue n-heptane (320 ml) was added, stirred for 3 hours, filtered and dried to give 470 g of title compound having purity 94% by HPLC.

### Example 11

### Purification of (R)—N-(1-naphthalen-1-yl-ethyl)-4-nitro-N-[3-(3-trifluoromethyl-phenyl)-propyl]-benzenesulfonamide

Method A: To a stirred solution of (R)—N-(1-naphthalen-1-yl-ethyl)-4-nitro-N-[3-(3-trifluoromethyl-phenyl)-propyl]-benzenesulfonamide (0.5 g) in dichloromethane (5 ml), silver nitrate (3 mg) was added and stirred the mixture for 24 hours. Demineralized water (5 ml) was added to the reaction mixture and layers were separated. The organic layer was washed with demineralized water (5 ml×2). The solvent was distilled off. The resulting product was crystallized with isopropanol (2 ml) to give 0.3 g of title compound having purity 99.28% by HPLC.

Method B: (R)—N-(1-naphthalen-1-yl-ethyl)-4-nitro-N-[3-(3-trifluoromethyl-phenyl)-propyl]-benzenesulfonamide of (12 g, having purity 95%) was dissolved in isopropanol (70 ml) at 85-90° C. The mixture was cooled to ambient temperature, filtered and dried under vacuum at 45-50° C. to give 10.6 g of title compound having purity 98.75% by HPLC.

Method C: To a stirred solution of (R)—N-(1-naphthalen-1-yl-ethyl)-4-nitro-N-[3-(3-trifluoromethyl-phenyl)-propyl]-benzenesulfonamide (440 g, having purity 94%) in dichloromethane and benzyltriethylammonium chloride (9.23 g, 0.041 mol), a solution of potassium permanganate (8.25 g, 0.0522 mol) in water (1.64 L) was added at 25° to 30° C. The reaction mixture was stirred for 24 hours at 25° to 30° C. and filtered through hyflo-bed. The dichloromethane solution was washed with water (1.0 L×2), treated with activated carbon (44 g) for 1 hour and filtered. Dichloromethane was distilled off under vacuum at 30° to 40° C. to afford the title compound. The resulting product was dissolved in isopropanol (1.32 L) at 85-90° C. The reaction mixture was cooled to 25-30° C., filtered and dried under vacuum at 40-45° C. to give 337 g of title compound as crystalline solid having purity 99.53% by HPLC.

### Example 12

### Preparation of Cinacalcet Free Base

A suspension of thiophenol (3.4 ml), potassium carbonate (8 g), acetonitrile (50 ml) and benzyltriethylammonium chloride (0.37 g) was stirred for 1 hour at ambient temperature. Thereafter, N-(1-naphthalen-1-yl-ethyl)-4-nitro-N-[3-(3-trifluoromethyl-phenyl)-propyl]-benzenesulfonamide (9 g) was added to the reaction mixture and heated at 60-70° C. for 5-8 hours. The solvent was distilled off. To the resulting residue, water (27 ml) and toluene (45 ml) were added and stirred. Layers were separated and toluene was distilled off to give title compound.

### Example 13

### Purification of Cinacalcet

To a solution of cinacalcet (4.2 g, having purity 85.0% by HPLC) in n-heptane (10 ml), silica gel (8.4 g) was added and the solvent was distilled off n-Heptane (40 ml) was added to above residue and stirred for 30 minutes. The mixture was filtered and the solvent was distilled off to obtain 2.52 g of title compound having purity 97.50% by HPLC.

### Example 14

### Preparation of Cinacalcet Hydrochloride

(R)—N-(1-Naphthalen-1-yl-ethyl)-4-nitro-N-[3-(3-trifluoromethylphenyl)propyl]benzenesulfonamide (300 g, 0.553 mol) to a stirred suspension of potassium carbonate (228.78 g, 1.655 mol), thiophenol (91.4 g, 0.83 mol), and benzyltriethylammonium chloride (12.58 g, 0.055 mol) in dimethylsulfoxide (900 ml) at 25° to 35° C. and stirred at 25° to 35° C. for 24 hours. Water (1.8 L) and isopropyl ether (1.5 L) were added to the reaction mixture and further stirred. Layers were separated and organic layer was washed with water (600 ml×2). Solvent was distilled off. To the resulting residue, ethyl acetate-hydrochloride (9%, 246 ml) was added at 0-5° C. followed by stirring at 20-25° C. for 2 hours. Thereafter, toluene (3 L) and water (1 L) were added to the mixture and stirred. Layers were separated and organic layer was distilled off at 60-65° C. under vacuum. Isopropyl ether (2.4 L) was added to the above residue and refluxed for further 24 hours. The reaction mixture was, then, cooled to ambient temperature, filtered, and dried to give 180 g of the title compound having purity 99.43% by HPLC.

### Example 15

### Purification of Cinacalcet Hydrochloride

Method A: Cinacalcet hydrochloride (2.5 g, having purity 97.5% by HPLC) was treated with diisopropyl ether: ethyl acetate mixture (12 ml, 9:1). The mixture was stirred and filtered to obtain 2.0 g of title compound having purity 99.7% by HPLC.

Method B: Cinacalcet hydrochloride (3.5 g, having purity 99.0% by HPLC) was stirred in diisopropyl ether: ethyl acetate mixture (1:1) for 1 hour and filtered to obtain 3 g of title compound having purity 99.8% by HPLC.

Method C: Cinacalcet hydrochloride (180 g, having purity 99.43%)) in isopropyl ether (1.44 L) was refluxed for 5 hours. The reaction mixture was cooled to ambient temperature, filtered and dried to give 177 g of the title compound having purity 99.72% by HPLC.

Method D: Cinacalcet hydrochloride (200 g, having purity 99.64%) was stirred in ethyl acetate: isopropyl ether (1:1, 1.05 L) at ambient temperature for 2 hours. The reaction mixture was filtered and dried under vacuum at 50° C. to give 190 g of the title compound having purity 99.81% by HPLC.

### Example 16

### Purification of Cinacalcet Hydrochloride

Method A: Cinacalcet hydrochloride (as prepared in example 7, method B) was stirred in diisopropyl ether-ethyl acetate mixture (9:1) for 1 hour and filtered to give 5 g of title compound having purity 99.77% and substituted carbamate impurity 0.07% by HPLC.

The resulting product was further stirred in diisopropyl ether-ethyl acetate mixture (1:1) for 1 hour and filtered to give 3.5 g of title compound having purity 99.81% and substituted carbamate impurity not detected by HPLC.

Method B: Cinacalcet hydrochloride (as prepared in example 7, method C) was stirred in diisopropyl ether-ethyl acetate mixture (9:1) for 1 hour and filtered to give 210 g of title compound having purity 99.7% and substituted carbamate impurity 0.03% by HPLC.

### Example-17

### Purification of Cinacalcet hydrochloride

Method A: Cinacalcet hydrochloride (5 g, having purity 96.5% and R-NEA 2.9% by HPLC) was dissolved in toluene (25 ml) and wash with aqueous hydrochloric acid (15 ml) followed by washing with water (15 ml×2) at a temperature of 40° C. Toluene was distilled off followed by addition of diisopropyl ether: ethyl acetate mixture (9:1). The mixture was stirred and filtered to obtain 4.5 g of title compound having purity 99% and R-NEA 0.03% by HPLC.

Method B: Cinacalcet hydrochloride (5 g, having purity 97.5% and R-NEA 2.0% by HPLC) was dissolved in toluene (25 ml) and wash with water (15 ml×2) at a temperature of 40° C. Toluene was distilled off followed by addition of diisopropyl ether: ethyl acetate mixture (9:1). The mixture was stirred and filtered to obtain 4.5 g of title compound having purity 99.6% and R-NEA 0.02% by HPLC.

### Example-18

### Purification of Cinacalcet Hydrochloride

To a solution of cinacalcet hydrochloride (5 g, having purity 98.0% by HPLC) in isopropylether (25 ml), was added 10% aqueous sodium carbonate solution (40 ml) and stirred for 1 hour. The organic layer was separated and washed with water (10 ml×2). Solvent was distilled off to obtain 4.2 g of cinacalcet. To a solution of above prepared cinacalcet (2.52 g) in tetrahydrofuran (13 ml) under nitrogen atmosphere, was added lithium aluminium hydride (54 mg) at 0 to −5° C. and stirred for 30 minutes. Ethyl acetate (3 ml) and methanol (3 ml) were added to reaction mixture and stirred for 30 minutes. The reaction mixture was filtered over celite and the solvents were distilled off. Isopropylether (10 ml) and 5N hydrochloric acid (4 ml) were added to above residue. The reaction mixture was stirred for 30 minutes, filtered, washed with water and dried to obtain 2.5 g of title compound having purity 98.59% by HPLC. The resulting product (2.0 g, having purity 98.59%) was stirred in diisopropyl ether: ethyl acetate (10 ml, 9:1). The mixture was filtered to obtain 1.7 g of the title compound having purity 99.8% by HPLC.

### Example-19

### Purification of Cinacalcet Hydrochloride

To a solution of cinacalcet hydrochloride (250 g, having purity 99.3% by HPLC) in isopropyl ether (1.25 L) at 25° C. was added 15% aqueous sodium carbonate solution and stirred for 2.0 hours. The organic layer was washed with water (250 ml×2), dried over sodium sulfate. The solvent was distilled off followed by addition of tetrahydrofuran (1.1 L) and cooled to 0° C. Lithium aluminium hydride (4.67 g) was added to the reaction mixture and stirred for 2 hours at 0° C., followed by addition of ethyl acetate (100 ml) and methanol (100 ml) at 0° C. Stirring was continued for 30 minutes and the reaction contents was filtered through hyflo bed and washed with methanol followed by distillation of the solvent. Isopropyl ether (1.1 L) and 5N hydrochloric acid (1.1 L) were added and stirred at 20-25° C. for 1.0 hour. The solid was filtered, washed with water (220 ml) and dried. The wet cake was dissolved in dichloromethane (1.1 L) and the aqueous layer was discarded. Active carbon (12 g) was added to dichloromethane layer and stirred at 40° C. for 30 minutes. The dichloromethane solution was filtered through hyflo bed and distilled in vacuum. Acetonitrile (1.1 l) was added and heated to 90° C. till complete dissolution followed by stirring for 3 hours at 20-25° C. The product was filtered, washed with acetonitrile and suck dried. The product was further dried at 45-50° C. in vacuum to obtain 216 g of title compound having purity 99.6% by HPLC.

### Example 20

### Preparation of Substituted Carbamate Impurity

To a solution of (R)-(1-naphthalen-1-yl-ethyl)-carbamic acid 3-(3-trifluoromethyl-phenyl)-propyl ester (2 g), dimethyl sulfoxide (10 ml), and sodium hydroxide (0.6 g) at 25-30° C., methanesulfonic acid-3-(3-trifluoromethyl-phenyl)-propyl ester (2.8 g) was added and stirred for 20 hours. Water (50 ml) was added to the reaction mixture and extracted with toluene (20 ml×3). Toluene layer was separated and distilled off to give 4 g of title compound having purity 44.18% by HPLC. The product was further purified by column chromatography to give title compound of purity 92.75% by HPLC.

1-36. (canceled)

37. A process for the preparation of cinacalcet of formula I, 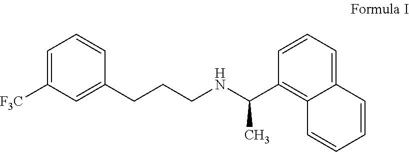
or a pharmaceutically acceptable salt thereof, which comprises the step of:
(a) providing a compound of formula II including isomers or mixture thereof; 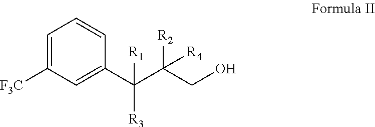
wherein R₁, R₂, R₃ and R₄ are hydrogen or R₁, R₂, together, form a double bond provided R₃ and R₄ are hydrogen or R₃, R₄, together, form a double bond provided R₁ and R₂ are hydrogen or R₁, R₂, R₃ and R₄ all are combined together to form triple bond;
(b) converting the hydroxyl group of compound of formula II into a good leaving group to obtain compound of formula III including isomers or mixture thereof; 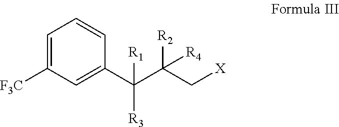
wherein R₁, R₂, R₃ and R₄ are as defined above and X is a good leaving group, by reaction in presence of activating agent and solvent;
(c) condensing the compound of formula III with the compound of formula Iv, 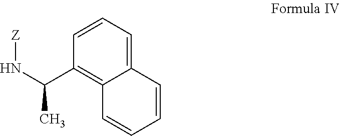
wherein Z is an amine protecting group and can be selected from allyl; substituted allyl; linear, branched or cyclic C₁₋₈ alkyl; substituted linear, branched or cyclic C₁₋₈ alkyl; linear, branched or cyclic C₁₋₈ alkenyl; substituted linear, branched or cyclic C₁₋₈ alkenyl; linear, branched or cyclic C₁₋₈ alkynyl; substituted linear, branched or cyclic C₁₋₈ alkynyl; —CN; —SO₂R″; —COOR″ wherein R″ can be alkyl, alkenyl, alkynyl, or aryl; —CONR′″R′″ wherein R″′ and R″″ can be same or different and individually selected from alkyl, alkenyl, alkynyl, or aryl; or and the like; all the above groups can be substituted at carbon with a group selected from alkyl, alkoxy or aryl and like,
in presence of a suitable base to prepare a compound of formula V; and 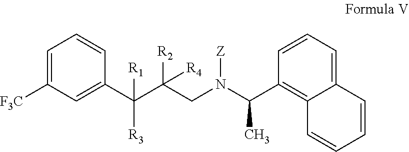
wherein R₁, R₂, R₃, R₄ and Z are as defined above.
(d) converting the compound of formula V to cinacalcet of formula I and pharmaceutically acceptable salts thereof.

38. The process according to claim 37, wherein in step b) activating agent is selected from thionyl halide, aliphatic or aromatic sulfonyl halide, phosphorous halides, phosphorous oxyhalide and the like; solvent includes water, halogenated solvents such as dichloromethane, chloroform; C₂₋₈ ether such as isopropyl ether, methyl tert-butyl ether; C₃₋₈ aromatic and aliphatic hydrocarbon such as toluene, xylene, ethyl benzene; C₂₋₅ nitrile such as acetonitrile; C₃₋₈ ketone such as acetone, ethyl methyl ketone; methyl isobutyl ketone; amide solvents such as dimethyl formamide, dimethylacetamide, methylpyrrolidone; and the like or mixture thereof; wherein in step c) base includes organic base and inorganic base and is selected from tertiary amines; RM or RMgX (wherein R can be alkyl or aryl and M can be alkali or alkaline earth metal); or alkoxide of alkali or alkaline earth metal; alkali or alkaline earth metal hydride, or hydroxide or carbonate or bicarbonate; or MNH₂ or MNSiR₇ (wherein M can be alkali metals and R₇ can be C₁₋₈ aliphatic or aromatic hydrocarbons and the like); or organometallic bases with or without additives.

39. The process according to claim 37, wherein step b) is carried out additionally in the presence of base, which is selected from organic base such as triethylamine, N,N-diisopropylethyl amine, N methylpyrrolidone or an inorganic base such alkali or alkaline metal hydroxide, carbonate, bicarbonate and the like or combination thereof, preferably triethylamine, sodium hydroxide, potassium hydroxide, sodium carbonate, potassium carbonate, sodium bicarbonate, lithium hydroxide and the like or combination thereof and wherein step c) is carried out in the presence of phase transfer catalyst which includes benzyl trimethylammonium chloride and bromide, cetyl trimethylammonium bromide, phosphonium compounds or synthetic resins, tetrabutylammonium bromide or chloride; benzyltriethylammonium chloride; tetrabutylammonium hydroxide; tricaprylmethylammonium chloride, dodecyl sulfate, sodium salt, such as sodium lauryl sulfate; tetrabutylammonium hydrogensulfate; hexadecyltributylphosphonium bromide; hexadecyltrimethyl ammonium bromide or resin amberlite IRA-410 and the like.

40. The process according to claim 37, wherein R₁, R₂, R₃ and R₄ are hydrogen.

41. The process according to claim 37, wherein the R₁, R₂ together, form a double bond provided R₃ and R₄ are hydrogen or R₃, R₄, together, form a double bond provided R₁ and R₂ are hydrogen and, wherein the R₁, R₂, R₃ and R₄ all are combined together to form triple bond.

42. A process for the preparation of cinacalcet of formula I and its pharmaceutically acceptable salts thereof which comprises the step of
a). providing a compound of formula IIa including isomers or mixture thereof; 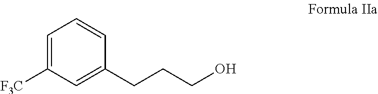
b). converting the hydroxyl group of compound of formula IIa into a good leaving group to obtain compound of formula IIIc including isomers or mixture thereof 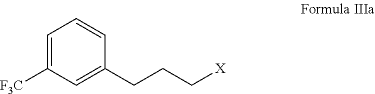
wherein X is a good leaving group
by reaction in presence of activating agent and solvent;
c). condensing the compound of formula IIIc with the compound of formula IV 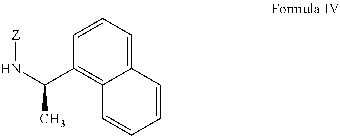
wherein Z is an amine protecting group and can be selected from allyl; substituted allyl; linear, branched or cyclic C₁₋₈ alkyl; substituted linear, branched or cyclic C₁₋₈ alkyl; linear, branched or cyclic C₁₋₈ alkenyl; substituted linear, branched or cyclic C₁₋₈ alkenyl; linear, branched or cyclic C₁₋₈ alkynyl; substituted linear, branched or cyclic C₁₋₈ alkynyl; —CN; —SO₂R″; —COOR″ wherein R″ can be alkyl, alkenyl alkynyl, or aryl; —CONR′″R′″ wherein R″′ and R″″ can be same or different and individually selected from alkyl, alkenyl, alkynyl, or aryl; or and the like; all the above groups can be substituted at carbon with a group selected from alkyl, alkoxy or aryl and like,
in presence of a suitable base to prepare a compound of formula Va; and 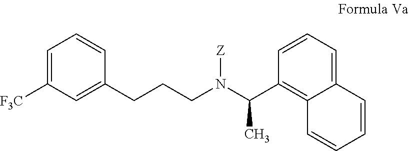
wherein Z is as defined above
d). converting the compound of formula Va to form cinacalcet of formula I and its pharmaceutically acceptable salts thereof.

43. The process according to claim 42, wherein in step b) activating agent is selected from thionyl halide, aliphatic or aromatic sulfonyl halide, phosphorous halides, phosphorous oxyhalide and the like, preferably thionyl bromide, thionyl chloride, methanesulfonyl chloride, benzenesulfonyl chloride, 4-nitrobenzenesulfonyl chloride or p-toluene sulfonyl chloride, phosphorus trichloride, phosphorous pentachloride, phosphorous oxychloride, phosphorous tribromide and the like; and solvent includes water, halogenated solvents such as dichloromethane, chloroform; C₂₋₈ ether such as isopropyl ether, methyl tert-butyl ether; C₃₋₈ aromatic and aliphatic hydrocarbon such as toluene, xylene, ethyl benzene; C₂₋₅ nitrile such as acetonitrile; C₃₋₈ ketone such as acetone, ethyl methyl ketone; methyl isobutyl ketone; amide solvents such as dimethyl formamide, dimethylacetamide, N methylpyrrolidone; and the like or mixture thereof; in step c) base includes organic base and inorganic base and is selected from tertiary amines; RM or RMgX (wherein R can be alkyl or aryl and M can be alkali or alkaline earth metal); or alkoxide of alkali or alkaline earth metal; alkali or alkaline earth metal hydride, or hydroxide or carbonate or bicarbonate; or MNH₂ or MNSiR₇ (wherein M can be alkali metals and R₇ can be C₁₋₈ aliphatic or aromatic hydrocarbons and the like); or organometallic bases with or without additives.

44. The process according to claim 42, wherein step b) is carried out additionally in the presence of base, which is selected from organic base such as triethylamine, N,N-diisopropylethyl amine, N-methylpyrrolidone or an inorganic base such alkali or alkaline metal hydroxide, carbonate, bicarbonate and the like or combination thereof, preferably triethylamine, sodium hydroxide, potassium hydroxide, sodium carbonate, potassium carbonate, sodium bicarbonate, lithium hydroxide and the like or combination thereof.

45. The process according to claim 42 wherein step c) is carried out in the presence of phase transfer catalyst which includes benzyl trimethylammonium chloride and bromide, cetyl trimethylammonium bromide phosphonium compounds or synthetic resins, tetrabutylammonium bromide or chloride; benzyltriethyl ammonium chloride; tetrabutylammonium hydroxide; tricapryl methylammonium chloride, dodecyl sulfate, sodium salt, such as sodium lauryl sulfate; tetrabutylammonium hydrogensulfate; hex adecyltributylphosphonium bromide; hexadecyltrimethyl ammonium bromide or resin amberlite IRA-410 and the like.

46. The process according to claim 42, wherein in step d) process for conversion of compound of formula Va to cinacalcet comprising the step of:
a). reacting the compound of formula Va with a suitable deprotecting agent;
b). optionally, isolating cinacalcet from the reaction mixture; and
c). converting the same to einacalcet pharmaceutically acceptable salts thereof.

47. The process according to claim 46, wherein step a) when Z is a tert-butoxycarbonyl group, then deprotecting agent is selected from strong acid; when Z is a p-nitrobenzene sulfonyl group, then deprotecting agent is selected from substituted or unsubstituted thiophenol, or samarium iodide, tributyltin hydride and the like.

48. A compound of formula V 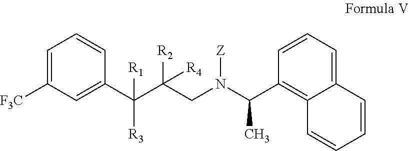
wherein R₁, R₂, R₃ and R₄ are hydrogen or R₁, R₉, together, form a double bond provided R₃ and R₄ are hydrogen or R₃, R₄, together, form a double bond provided R₁ and R₂ are hydrogen or R₁, R₂, R₃ and R₄ all are combined together to form triple bond; and
wherein Z is an amine protecting group and can be selected from allyl; substituted allyl; linear, branched or cyclic C₁₋₈ alkyl; substituted linear, branched or cyclic C₁₋₈ alkyl; linear, branched or cyclic C₁₋₈ alkenyl; substituted linear, branched or cyclic C₁₋₈ alkenyl; linear, branched or cyclic C₁₋₈ alkynyl; substituted linear, branched or cyclic C₁₋₈ alkynyl; —CN; —SO₂R″; —COOR″ wherein R″ can be alkyl, alkenyl, alkynyl, or aryl; —CONR″′R′″ wherein R′″ and R″″ can be same or different and individually selected from alkenyl, alkynyl, or aryl; or and the like; all the above groups can be substituted at carbon with a group selected from alkyl, alkoxy or aryl and

49. The compound according to claim 48, wherein R₁, R₂, R₃ and R₄ are hydrogen has structure of formula Va 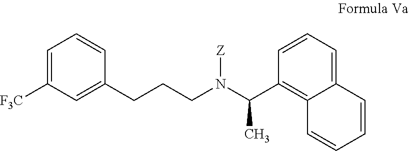
wherein Z is as defined above.

50. The compound according to claim 49, wherein Z is selected amongst carbobenzyloxy, p-methoxybenzyl carbonyl, tert-butyloxycarbonyl, 9-fluorenylmethyloxycarbonyl, benzyl, p-methoxybenzyl, 3,4-dimethoxybenzyl, benzyloxycarbonyl group, p-methoxyphenyl, tert-butyldimethylsilyl; other sulfonyl such as p-nitrobenezensulfonyl, methanesulfonyl, p-toluenesulfonyl, benzenesulfonyl group, and the like

51. A substituted carbamate impurity of cinacalcet, having formula VII.

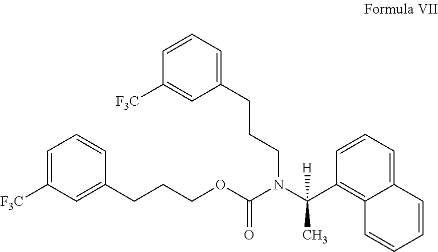

52. An isolated substituted carbamate impurity of claim 51.

53. Cinacalcet hydrochloride having substituted carbamate impurity of claim 51, in an amount of about 0.03 area percent to about 0.15 area percent.

54. The process for the preparation of cinacalcet hydrochloride having substituted carbamate impurity of formula VII less than 0.15% according to claim 53, comprises:
a). reacting a compound of formula IV, 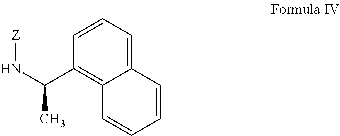
wherein Z is selected from a functional group of general formula —COOR″ wherein R″ is selected from straight chain or branched C₁₋₈ alkyl group;
substituted or unsubstituted aryl group
with a compound of formula IIIa, 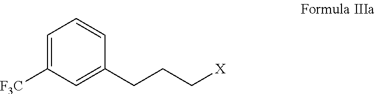
wherein X is a good leaving group
in the presence of a base in a solvent to form a compound of formula Va, 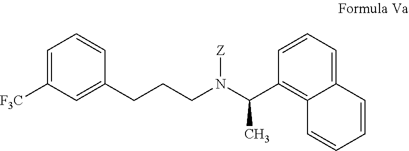
wherein Z is as defined above
having substituted carbamate impurity of formula VII;
b). treating the compound of formula Va with a source of hydrogen chloride in a solvent to form cinacalcet hydrochloride;
c). purifying the cinacalcet hydrochloride with a suitable solvent; and
d). isolating cinacalcet hydrochloride having substituted carbamate impurity of formula VII less than 0.15% by HPLC.

55. The process according to claim 54, wherein in step a) base include organic base or inorganic base such as C₁₋₈ trialkylamine, alkali or alkaline metal hydroxide, alkoxide, carbonates, bicarbonates thereof; and solvent include ketones such as methyl isobutyl ketone, methyl ethyl ketone; ethers such as isopropyl ether, methyl tertiary butyl ether; nitriles such as acetonitrile; halogenated solvents such as chloroform; dimethyl sulfoxide; and the like or mixture thereof.

56. The process according to claim 54, wherein in step b) source of hydrogen chloride include aqueous hydrochloric acid, hydrogen chloride gas or mixture thereof with suitable solvent selected from alcohol, ester, ether and the like; in step c) suitable solvent include ester such as ethyl acetate; ethers such as diisopropyl ether, methyl tertiary butyl ether; aliphatic hydrocarbon such as n-heptane and the like or mixture thereof in any suitable proportion.
